(19) United States
(12) Patent Application Publication
(10) Pub. No.: US 2026/0256524 A1
(43) Pub. Date: Sep. 3, 2026

(54) SYSTEMS AND METHODS FOR SINGLE POSITION 360 DEGREE CERVICAL FUSION

(71) Applicant: Warsaw Orthopedic, Inc., Warsaw, IN (US)

(72) Inventors: Adam D. Glaser, Collierville, TN (US); Ping Li, Germantown, TN (US); Newton H. Metcalf, Jr., Memphis, TN (US)

(21) Appl. No.: 19/489,191

(22) PCT Filed: Jun. 13, 2024
(86) PCT No.: PCT/US2024/033908

§ 371 Date: Dec. 1, 2025

Related U.S. Application Data
(60) Provisional application No. 63/522,848, filed on Jun. 23, 2023.
(60) Provisional application No. 63/656,692, filed on Jun. 6, 2024.

(51) Int. Cl. A61B 34/20; A61B 34/10; A61B 34/30; A61B 90/00

(52) U.S. Cl. CPC A61B 34/20; A61B 34/10; A61B 34/30; A61B 90/361; A61B 90/37; A61B 2034/107; A61B 2034/2055; A61B 2034/303

(57) ABSTRACT

A method of single position 360 degree cervical fusion includes positioning a patient on a surgical table in a lateral position, orienting and retaining a spine of the patient in a desired sagittal alignment, performing anterior cervical disc fusion (ACDF), reorienting and retaining a head of the patient to facilitate posterior access, and performing posterior fixation. Devices and systems configured to facilitate performing the single position 360 degree cervical fusion method are also provided.

### CROSS-REFERENCE TO RELATED APPLICATIONS

This application claims the benefit of, and priority to, U.S. Provisional Patent Application No. 63/522,848, filed on Jun. 23, 2023, and U.S. Provisional Patent Application No. 63/656,692, filed on Jun. 6, 2024, the entire contents of each of which is hereby incorporated herein by reference.

### FIELD

The present disclosure relates to spinal surgery and, more particularly, to systems and methods for single position 360 degree cervical fusion.

### BACKGROUND

Currently, 360 degree cervical fusion is a multi-step procedure wherein an anterior cervical disc fusion (ACDF) is first performed with the patient in the supine position. An ACDF typically involves performing a discectomy within an intervertebral disc space, inserting a bone graft into the intervertebral disc space, and attaching a surgical plate on the anterior side of the vertebral column to span the intervertebral disc space and provide stability while spinal fusion occurs. Upon completion of the ACDF, the patient is repositioned in the prone position. With the patient in the prone position, screws are secured to the vertebrae to provide additional stability while the spinal fusion occurs.

In more complex cervical fusion procedures, a 540 degree approach (where the patient is repositioned from supine, to prone, and back to supine during the procedure) or 720 degree approach (where the patient is repositioned from supine, to prone, back to supine, and again to prone during the procedure) may be required.

The time required to complete 360 degree (and, even more so, 540 degree and 720 degree) cervical fusion as currently performed is significant, including approximately 30 to 60 minutes devoted to repositioning the patient, e.g., from the supine position to the prone position. In addition, there is the potential for harming the patient during the repositioning(s).

### SUMMARY

Terms including “generally,” “about,” “substantially,” and the like, as utilized herein, are meant to encompass variations, e.g., manufacturing tolerances, material tolerances, use and environmental tolerances, measurement variations, design variations, and/or other variations and tolerances, up to and including plus or minus 10 percent. Further, any or all of the aspects described herein, to the extent consistent, may be used in conjunction with any or all of the other aspects described herein.

Provided in accordance with aspects of the present disclosure is a method of single position 360 degree cervical fusion. The method includes positioning a patient on a surgical table in a lateral position, orienting and retaining a spine of the patient in a desired sagittal alignment, performing anterior cervical disc fusion (ACDF), reorienting and retaining a head of the patient to facilitate posterior access, and performing posterior fixation.

Performing the posterior fixation may include transfacet screw fixation or any other suitable posterior fixation such as, for example, lateral mass screw fixation or pedicle screw fixation.

In aspects where transfacet screw fixation is performed, such may be performed with or without fusion. The fusion, where performed, may include removing facet joint cartilage, decorticating a facet joint surface, and/or delivering a flowable orthobiologic to a transfacet joint space.

In an aspect of the present disclosure, at least a portion of the ACDF is performed by a surgeon with handheld tools, and at least a portion of the posterior fixation is performed by a surgical robot and/or using robotically guided tool(s). In such aspects, the surgeon may be positioned on an anterior side of the patient (for the at least a portion of the ACDF) and the surgical robot may be positioned on a posterior side of the patient (for the at least a portion of the posterior fixation).

In another aspect of the present disclosure, the ACDF includes installing an anterior plate retained by a plurality of screws.

In still another aspect of the present disclosure, a navigation component is attached to the anterior plate and utilizes during the posterior fixation, e.g., to facilitate trajectory planning and/or navigation.

In yet another aspect of the present disclosure, the method further includes airplaning the surgical table before performing the ACDF and/or before performing the posterior fixation.

In still yet another aspect of the present disclosure, a manipulation apparatus attached to the head of the patient is utilized to facilitate orienting the spine of the patient, retaining the spine of the patient, reorienting the head of the patient, and retaining the head of the patient.

In another aspect of the present disclosure, positioning the patient on the surgical table in the lateral position includes extending an upward arm of the patient along a body of the patient and extending a downward arm of the patient anteriorly from the body and bent at the elbow such that a lower portion of the downward arm of the patient extends cephalad.

In another aspect of the present disclosure, the posterior fixation includes at least one of planning a trajectory of at least one screw or installing at least one screw using navigation.

In yet another aspect of the present disclosure, performing the ACDF includes retracting a wound with first and second retractor blades arranged in a vertical orientation.

In still another aspect of the present disclosure, the method further includes providing additional support to a posterior side of the patient during at least a portion of the ACDF. The additional support may be removed after the at least the portion of the ACDF.

In another aspect of the present disclosure, positioning the patient on the surgical table includes positioning the patient offset from a longitudinal axis of the surgical table.

In still yet another aspect of the present disclosure, positioning the patient on the surgical table includes securing the patient relative to the surgical table.

Devices and systems configured to facilitate performing some or all of the aspects of the above-detailed single position 360 degree cervical fusion are also provided in accordance with the present disclosure.

Also provided in accordance with the present disclosure is a manipulation apparatus configured to facilitate positioning and maintaining a spine of a patient. The manipulation apparatus includes a first frame assembly and a second frame assembly. The first frame assembly includes a base frame configured to engage a torso of a patient to fix the base frame relative to the torso of the patient. The second frame assembly is coupled to the first frame assembly and includes a support frame and a head frame coupled to the support frame and configured to engage a head of the patient to fix the head frame relative to the head of the patient. The support frame is movable relative to the base frame in at least two degrees of freedom and the head frame is movable relative to the support frame in at least one degree of freedom.

In an aspect of the present disclosure, the support frame is configured to pivot relative to the base frame to enable pivoting of the head and neck of the patient relative to the torso of the patient and to translate relative to the base frame to enable extension or compression of the head and neck of the patient relative to the torso of the patient.

In another aspect of the present disclosure, the support frame is configured to lock relative to the base frame to at least one of retain a position of the head and neck of the patient relative to the torso of the patient or retain an extension or compression of the head and neck of the patient relative to the torso of the patient.

In another aspect of the present disclosure, the head frame is configured to pivot relative to the support frame to enable pivoting of the head of the patient anteriorly or posteriorly relative to the neck of the patient.

In still another aspect of the present disclosure, the head frame is configured to lock relative to the support frame to retain the head of the patient relative to the neck of the patient.

In yet another aspect of the present disclosure, the second frame assembly further includes an outer frame coupling the support frame with the first frame assembly.

In still yet another aspect of the present disclosure, the first frame assembly further includes a pair of linkages coupling the outer frame with the base frame.

In another aspect of the present disclosure, the outer frame includes at least one mounting extension configured to enable mounting of the outer frame to a patient support. In such aspects, the outer frame may include a plurality of mounting extensions with at least one mounting extension of the plurality of mounting extensions configured to enable mounting of the outer frame to a patient support in an offset position.

In yet another aspect of the present disclosure, the manipulation apparatus further includes an adjustable bolster assembly coupled to the second frame assembly. The adjustable bolster assembly includes a bolster movable relative to the second frame assembly.

A surgical periscope provided in accordance with the present disclosure includes a body defining a first end portion and a second end portion. The first end portion of the body is configured to operably couple to an objective end of a surgical microscope having a first viewing direction. The second end portion of the body is disposed at an angle relative to the first end portion of the body and has a second viewing direction disposed at an angle relative to the first viewing direction. At least one optical element is disposed within the body and configured to relay a field of view in the second viewing direction to the objective end of the surgical microscope to enable visualization of the field of view through the surgical microscope.

In an aspect of the present disclosure, the first end portion of the body is configured to releasably engage the surgical microscope at the objective end of the surgical microscope.

In another aspect of the present disclosure, the first end portion of the body is physically connected to the second end portion of the body or wirelessly connected to the second end portion of the body.

In still another aspect of the present disclosure, the second viewing direction is disposed at an angle of about 90 degrees relative to the first viewing direction A support assembly for supporting a patient in a lateral position on a surgical table in accordance with the present disclosure is provided including a mount configured for positioning on a surgical table and a first support rotatably coupled to the mount. The first support includes a body having a plurality of independently adjustable sections to enable independent adjustment of a height of each of the plurality of independently adjustable sections.

In an aspect of the present disclosure, the support assembly further includes a strap attached to the first support and configured to strap a head of a patient to the first support.

In another aspect of the present disclosure, the support assembly further includes g to claim 35, further comprising a second support pivotably coupled to the first support, the second support defining an elongate configuration.

A method of single position 360 degree cervical fusion provided in accordance with the present disclosure includes positioning a patient on a surgical table in a lateral position, performing anterior cervical disc fusion (ACDF), and performing posterior fixation. The posterior fixation includes, from a first approach to a facet joint, preparing for insertion of an insert into the facet joint or inserting the insert into the facet joint. The posterior fixation further includes, from a second, different approach to the facet joint, performing transfacet screw fixation.

In aspects of the present disclosure, the method may include any of the features detailed with respect to the above methods, and vice versa.

In another aspect of the present disclosure, at least one first instrument is used for the first approach and at least one second instrument is used for the second approach. The first and second instruments, in aspects, are coupled to one another in at least one degree of freedom.

In yet another aspect of the present disclosure, the first approach is adjacent to opposing bone surfaces of the facet joint and the second approach is transverse to the facet joint.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a perspective view of a surgical operating room configured for performance of a single position 360 degree cervical fusion procedure in accordance with aspects of the present disclosure;

FIG. 2 is a flow diagram of a method of performing a single position 360 degree cervical fusion procedure in accordance with aspects of the present disclosure;

FIG. 3 is a flow diagram of a first portion of the method of FIG. 2 in accordance with aspects of the present disclosure;

FIG. 4 is a top, schematic illustration of a patient disposed on a surgical table in a lateral position for performing the single position 360 degree cervical fusion procedure in accordance with aspects of the present disclosure;

FIG. 5A is a side, anterior, schematic illustration of the patient disposed on the surgical table in the lateral position for performing the single position 360 degree cervical fusion procedure in accordance with aspects of the present disclosure;

FIG. 5B is a side, perspective view of another surgical table configured for performing the single position 360 degree cervical fusion procedure in accordance with aspects of the present disclosure;

FIG. 6 is an enlarged, side view illustrating the patient's chin disposed in an upward orientation such that the patient's head is in extension in accordance with aspects of the present disclosure;

FIG. 7A is a schematic illustration of the patient including a manipulation apparatus secured to the patient to enable adjustment of the position of the patient's head and/or neck in accordance with aspects of the present disclosure;

FIGS. 7B and 7C are front perspective and side views, respectively, of another a manipulation apparatus secured to the patient to enable adjustment of the position of the patient's head and/or neck in accordance with aspects of the present disclosure;

FIG. 8 is a flow diagram of a second portion of the method of FIG. 2;

FIGS. 9 and 10 are images taken during performance of an ACDF in accordance with aspects of the present disclosure;

FIG. 11 is a perspective view illustrating disc replacement during performance of the ACDF in accordance with aspects of the present disclosure;

FIGS. 12 and 13 are other images taken during performance of the ACDF in accordance with aspects of the present disclosure;

FIG. 14 is an x-ray image illustrating a portion of the patient's spine after completion of the ACDF in accordance with aspects of the present disclosure;

FIG. 15 is a flow diagram of a third portion of the method of FIG. 2;

FIG. 16 is an enlarged, side view of the patient with the patient's chin disposed in a downward orientation such that the patient's head is in flexion in accordance with aspects of the present disclosure;

FIG. 17 is an image taken during preparation for robotic transfacet screw fixation in accordance with aspects of the present disclosure;

FIGS. 18 and 19 are screenshots illustrating robotic trajectory planning for transfacet screw fixation in accordance with aspects of the present disclosure;

FIGS. 20A-20E are perspective views progressively illustrating tool positioning and utilization during transfacet screw fixation in accordance with aspects of the present disclosure;

FIGS. 21-23 are x-ray images illustrating the left side transfacet screws, plate and screws of the ACDF, and right side transfacet screws, respectively, after completion of the single position 360 degree cervical fusion procedure in accordance with aspects of the present disclosure;

FIGS. 24A and 24B are side views of a coupling assembly provided in accordance with the present disclosure wherein first and second portals of the coupling assembly are disposed in first and second different positions relative to one another, respectively;

FIG. 25 illustrates the coupling assembly of FIGS. 24A and 24B in use facilitating guiding one or more surgical tools and/or materials to spinal anatomy of a patient, e.g., a facet joint or bone surface;

FIG. 26 is an enlarged view of spinal anatomy of a patient including an insert disposed within the facet joint and a screw extending through the facet joint and engaged with the insert;

FIGS. 27A and 27B are cross-sectional views of the screw and insert of FIG. 26 disengaged from one another and engaged with one another, respectively;

FIG. 28 illustrates a surgical periscope provided in accordance with the present disclosure shown providing ergonomic microscope visualization of spinal anatomy of a patient;

FIG. 29 is an enlarged view of a distal portion of the surgical periscope of FIG. 28 shown providing ergonomic microscope visualization of spinal anatomy of a patient;

FIG. 30 is a schematic illustration of a visualization system provided in accordance with the present disclosure shown providing ergonomic visualization of spinal anatomy of a patient;

FIG. 31 is a top, schematic illustration of a patient disposed on a surgical table in the lateral position supported by a plurality of support apparatus;

FIG. 32 is a side, schematic illustration of the plurality of support apparatus of FIG. 31 supporting the patient on the surgical table in the lateral position;

FIG. 33 is a side view of an adjustable head and neck support and an adjustable arm support of the plurality of support apparatus of FIG. 31;

FIG. 34 is a side view of a posterior bolster of the plurality of support apparatus of FIG. 31;

FIGS. 35 and 36 are top views of another manipulation apparatus provided in accordance with the present disclosure;

FIGS. 37A and 37B are side views of the manipulation apparatus of FIGS. 35 and 36;

FIGS. 38A and 38B are front views of an adjustable bolster assembly of the manipulation apparatus of FIGS. 35 and 36;

FIGS. 39A-39C illustrate preparation for and performing facet joint fixation in accordance with aspects of the present disclosure using a screw and an insert installed from a common access location;

FIGS. 40A and 4B illustrate cannulae configured to facilitate preparation for and performing facet joint fixation in accordance with other aspects of the present disclosure from a common access location;

FIGS. 41A-41C illustrate preparation for and performing facet joint fixation in accordance with still other aspects of the present disclosure using a screw and an insert installed from a common access location;

FIG. 42 illustrates preparation for and performing facet joint fixation in accordance with yet other aspects of the present disclosure using a screw and an insert installed from different access locations;

FIG. 43 illustrates preparation for and performing facet joint fixation in accordance with still yet other aspects of the present disclosure using a screw and an insert installed from different access locations; and FIGS. 44A and 44B illustration preparation for facet joint fixation in accordance with the present disclosure from different approach directions and with different tools.

### DETAILED DESCRIPTION

The systems and methods of the present disclosure enable performance of a single position 360 degree cervical fusion procedure, thus significantly reducing the time to complete the procedure (e.g., by obviating the need to reposition the patient) and reducing risks associated with the procedure (e.g., the potential for harming the patient during repositioning(s)). Further, suitable clearance, visibility, and working space for both a surgeon and a surgical robot are provided, thus facilitating performance of the 360 degree cervical fusion.

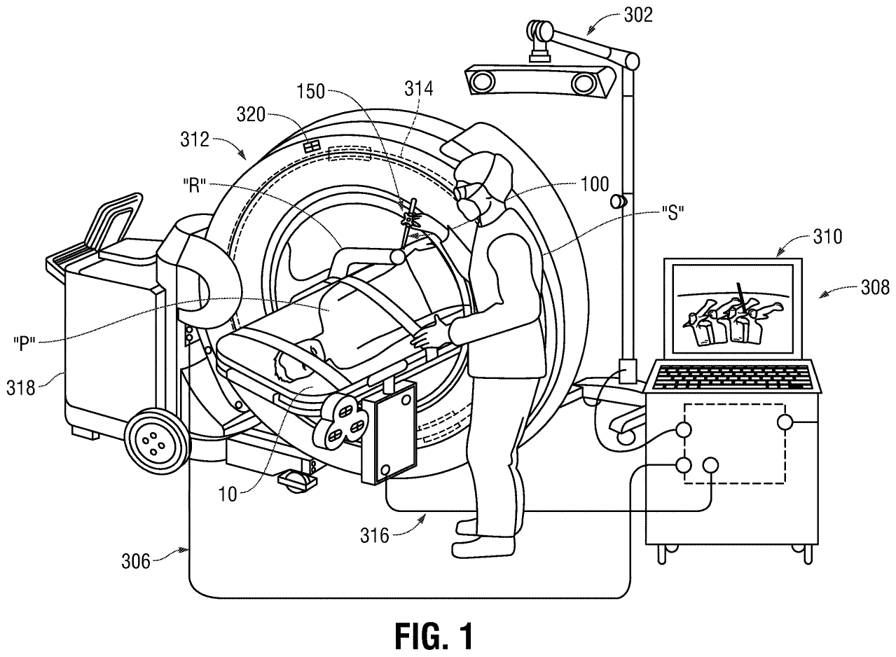

Referring to FIG. 1, a surgical operating room is shown configured for performance of a single position 360 degree cervical fusion procedure in accordance with the present disclosure. The surgical operating room includes a patient “P” disposed on a surgical table 10, a surgeon “S,” a surgical robot “R,” a plurality of surgical tools 100, 150 (e.g., any of the tool detailed herein), and a surgical navigation system 306.

Surgical navigation system 306, in aspects, includes an imaging device 312 such as, for example, an x-ray imaging device. Imaging device 312 may define an annularly extending housing that encloses an image capture assembly 314. Image capture assembly 314 may include an x-ray source (or other imaging emission portion) and an x-ray receiver (or other imaging receiver portion) mounted in substantial opposition to one another on a track of image capture assembly 314. The source and receiver of image capture assembly 314 are operable to rotate, e.g., 360 degrees, through the annularly extending housing of imaging device 312 during image acquisition, thereby rotating the source and receiver of image capture assembly 314 along the track of image capture assembly 314 to enable capture of image data at various different circumferential locations. More specifically, the imaging components of image capture assembly 314 may rotate around a central point or axis, allowing image data of the patient “P” to be acquired from multiple directions and/or in multiple planes. As such, imaging device 312 collects suitable image data to enable generation of various views and/or a three-dimensional model, e.g., a three-dimensional x-ray model. The views and/or three-dimensional model may be utilized to facilitate surgical planning, e.g., for determining an end position, angle, depth, and/or trajectory of surgical tools such as, for example, surgical instrumentation, surgical hardware, etc. relative to one another and/or patient anatomy.

In aspects, surgical navigation system 306 may be configured for fluoroscopic imaging to generate substantially real-time images, e.g., three-dimensional images, of the patient “P.” In such configurations, a position of image capture assembly 314 can be used in conjunction with a tracking system 316 to determine the position of image capture assembly 314 and the image data relative to the patient “P.” Tracking system 316 can include various portions that are associated or included with surgical navigation system 306. Tracking system 316 may include, for example, an optical tracking system and/or an electromagnetic (EM) tracking system. One or more tracking devices can be tracked in real-time using tracking system 316 and the information used by surgical navigation system 306 to display, in the real-time three-dimensional images, a position of one or more items (e.g., a patient tracking device, an imaging device tracking device 320, a surgical tool tracking device, etc.) to allow such items to be tracked relative to one another, the patient “P,” and/or other components.

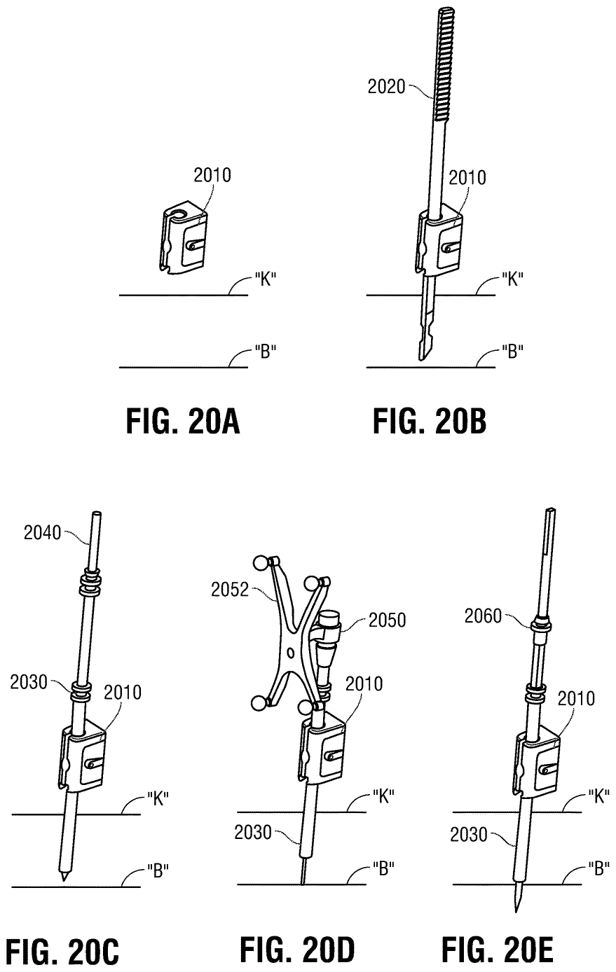

Continuing with reference to FIG. 1, a navigation component including an emitter array 304 (FIG. 13) configured to generate a signal capable of being detected by a sensor array 302 of surgical navigation system 306 is fixed relative to the patient “P” to enable determination of a known reference position relative to the patient “P,” e.g., in three-dimensional space. One or more of the surgical tools 100, 150 may also include emitter array (see, e.g., component 2052 (FIG. 20D)) configured to generate a signal capable of being detected by sensor array 302 of surgical navigation system 306 such that, in conjunction with the known reference position, a three-dimensional spatial position and/or a trajectory of the one or more surgical tools 100, 150 relative to the patient “P” can be displayed and/or tracked to facilitate surgical planning and/or navigation.

More specifically, image data, e.g., x-ray image data, obtained using the one or more imaging devices 312 is transmitted to a computer 318 where the image data may be forwarded to computer 308 for saving the image data, generating images from the image data (e.g., various 2D-images, a 3D model, etc.), digitally manipulating the generated images, printing the generated images, and/or displaying the image data (e.g., on monitor 310). With respect to display in particular, a processor of computer 308 of surgical navigation system 306 is configured to output, for display on monitor 310, the image data along with real-time tracking of the position of one or more surgical tools 100, 150 (including surgical instrumentation, surgical hardware (e.g., screws, plates, rods, etc.), and/or other suitable surgical tools) relative to one another, the patient “P,” and/or other suitable reference point(s).

Thus, as detailed above, surgical navigation system 306 may enable surgical planning, e.g., the determination and mapping of locations, positions, angles, depths, trajectories, etc. of one or more surgical tools, and/or may facilitate navigation of the one or more surgical tools during use, e.g., according to a determined surgical plan. Although exemplary tracking and imaging devices are detailed above, other suitable tracking and/or imaging devices are also contemplated.

Surgical robot “R” may include one or more robotic arms each configured to hold and/or manipulate one or more surgical tools. Each robotic arm of surgical robot “R” may further include position sensors configured to enable determination of the location and/or orientation of the one or more surgical tools connected thereto. Each robotic arm of surgical robot “R” may be actively driven by a surgeon to manipulate the corresponding surgical tool or may be automatically driven based upon instructions to perform a particular surgical task or portion thereof. Such instructions may include, for example, manipulating a surgical tool along a determined trajectory, to a determined location, to a determined depth, etc. and/or actuating, deploying, driving, etc. the surgical tool a determined amount and/or to a determined location.

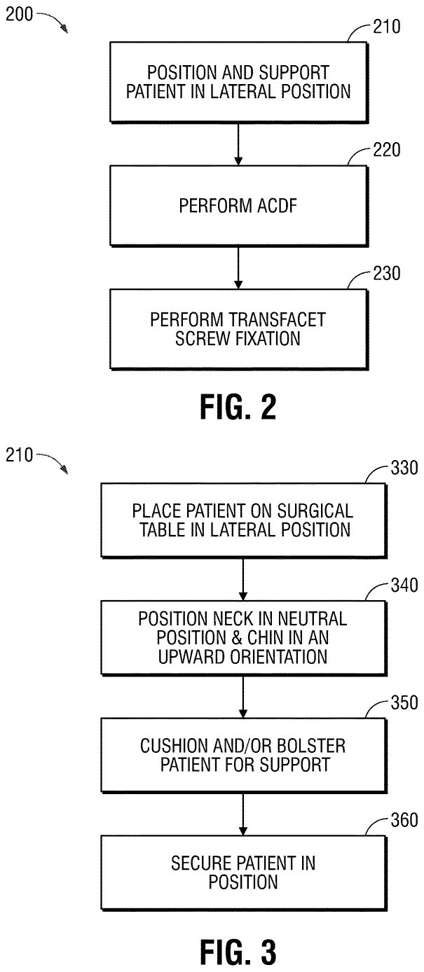

With additional reference to FIG. 2, a method of single position 360 degree cervical fusion provided in accordance with the present disclosure is shown generally identified by reference numeral 200. Although reference is made herein to the configuration of the surgical operating room of FIG. 1, any other suitable surgical operating room configuration is also contemplated.

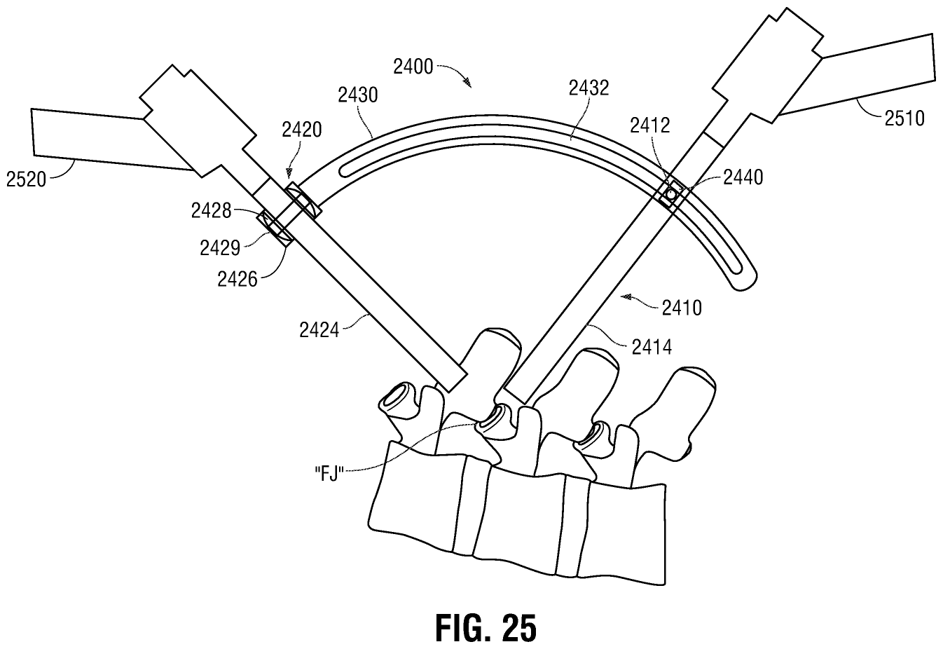

Single position 360 degree cervical fusion method 200 includes positioning and supporting a patient in a lateral position (as indicated at 210), performing anterior cervical disc fusion (ACDF) (as indicated at 220), and performing transfacet screw fixation (as indicated at 230). As an alternative or in addition to transfacet screw fixation, any other suitable posterior fixation may be performed such as, for example, lateral mass screw fixation or pedicle screw fixation. Thus, although detailed herein with respect to transfacet screw fixation, it is understood that other suitable posterior fixations may be performed. Further, the transfacet screw fixation (or other suitable posterior fixation) may be performed with or without fusion and may include removing facet joint cartilage, decorticating a facet joint surface, and/or delivering a flowable orthobiologic to a transfacet joint space. Decorticating the facet joint, in aspects, may be performed from multiple trajectories (see, e.g., FIG. 25) using, for example, a surgical burr, rasp, and/or other suitable tools depending upon the trajectory(s) utilized.

Each of portions 210, 220, 230 of method 200, along with components and systems configured to facilitate performance of method 200, are described in detail, in turn, hereinbelow. Any or all of portions 210, 220, 230 of method 200 may be performed with the assistance of imaging, e.g., fluoroscopic imaging, and/or surgical navigation, such as detailed above.

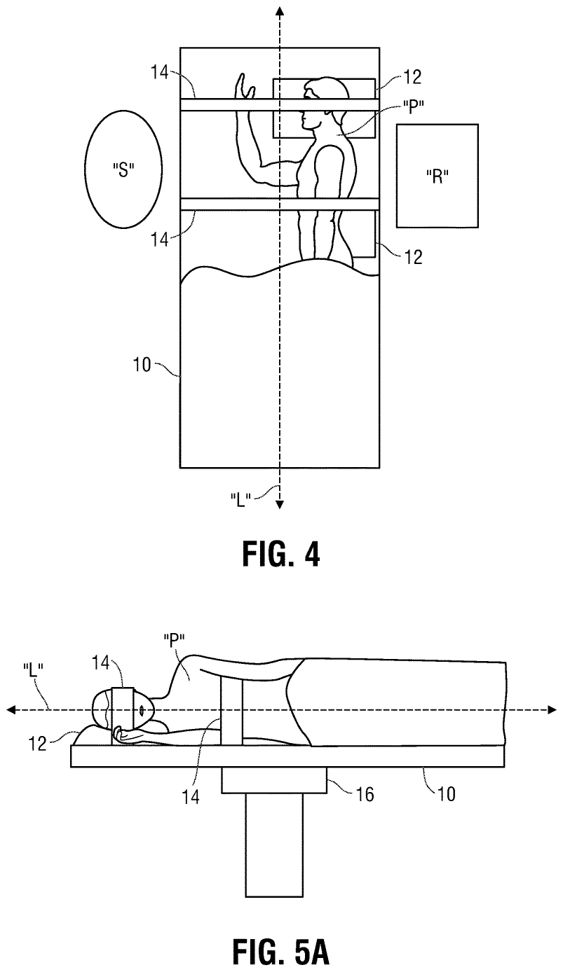

Turning to FIGS. 3-6, positioning and supporting the patient in the lateral position 210 (see also FIG. 2) initially includes placing the patient “P” on surgical table 10 in a lateral position, as indicated at 330. The patient “P” may be placed in a left or right lateral position, depending upon, for example, surgeon preference, anatomical considerations, and/or other factors. The surgeon “S” is disposed on the anterior side of the patient “P” while the surgical robot “R” is disposed on the posterior side of the patient “P.” If necessary, the patient “P” may be positioned laterally off center from a longitudinal axis “L” of the surgical table 10 (extending between the head and foot ends of surgical table 10) such as, for example, closer to surgical robot “R” to ensure reachability of the arm of the surgical robot “R,” e.g., to facilitate performance of the transfacet screw fixation 230 (FIG. 2).

In addition, placing the patient “P” on surgical table 10 in the lateral position, as indicated at 330, includes positioning the upward arm of the patient “P” to extend along the upward side of the body of the patient “P,” while the downward arm of the patient “P” is positioned to extend anteriorly from the body of the patient “P” and bent at the elbow such that the lower portion of the downward arm of the patient “P” extends cephalad. This configuration facilitates access to the patient “P” from, in particular, the anterior side to facilitate performing ACDF and avoiding collision between the surgeon “S” and/or surgical tools with the arms of the patient “P.” However, other arm positionings are also contemplated.

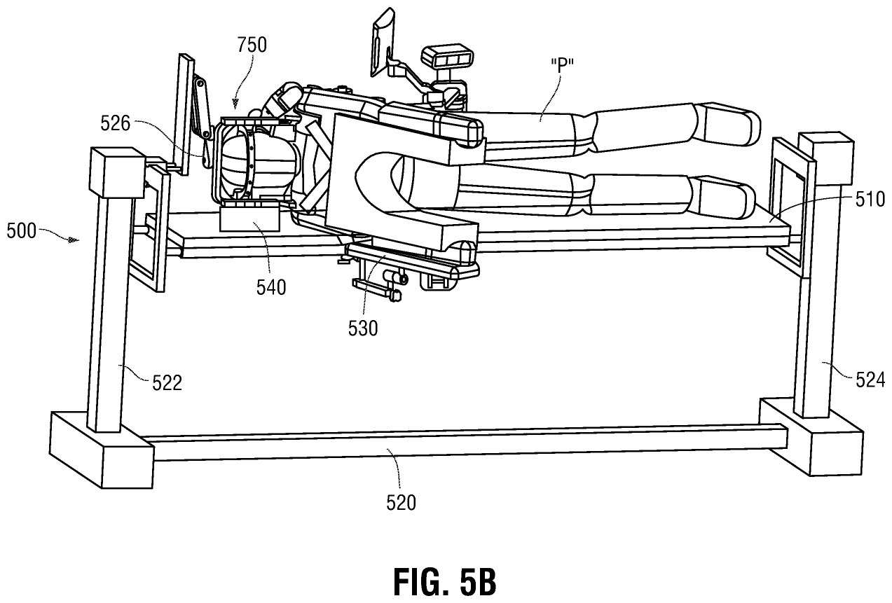

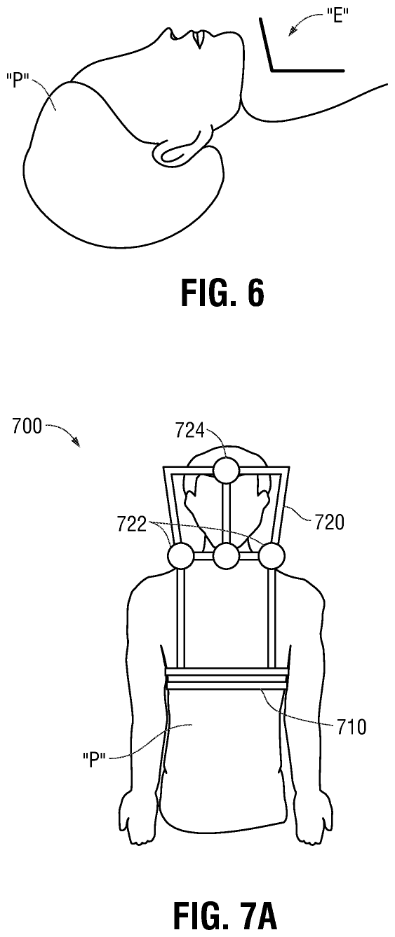

Once the patient “P” is positioned on table 10 at 230, as detailed above, or in conjunction with positioning the patient “P” on table 10 at 230, the spine of the patient “P” is oriented in a desired sagittal alignment. As indicated at 340, for example, a desired sagittal alignment may be achieved by positioning the head and neck of the patient “P” as necessary to achieve a neutral position to maintain normal cervical lordosis (see FIG. 5A). In addition, as also indicated at 340, achieving the desired sagittal alignment may include tilting the chin of the patient “P” upward, e.g., such that the head of the patient “P” is in extension “E” to provide sufficient anterior clearance (see FIG. 6) for performing the ACDF without interference from the head of the patient ‘P.” Imaging, e.g., fluoroscopy, utilizing, for example, surgical navigation system 316 (FIG. 1), may be utilized to facilitate alignment of the patient “P” and/or to confirm proper alignment of the patient “P.”

In order to facilitate positioning the patient “P” as detailed above and/or in order to retain the above-detailed positioning of the patient “P,” supports 12 such as cushions (e.g., pillows), bolsters, and/or other suitable supports are utilized as necessary, as indicated at 350.

Once the patient “P” is properly positioned on surgical table 10, supported, and/or bolstered as necessary (see, e.g., 330-350), the patient “P” is secured in position, as indicated at 360. The patient “P” may be secured in position using any suitable restraints 14 such as, for example, straps, tape, mechanical framing, and/or any other suitable components or combinations thereof.

Before, after, or in conjunction with placing, positioning, and securing the patient “P” as detailed above, drapes and/or other sterile barriers are utilized to properly prepare the sterile surgical field. In addition, intubation is completed, if not done so already.

Referring in particular to FIG. 5A, in aspects, in order to facilitate positioning the patient “P” to perform the ACDF, surgical table 10 may include an adjustment mechanism 16 configured to tilt the patient supporting surface of surgical table 10. For example, adjustment mechanism 16 may enable airplaning (tilting side-to-side about longitudinal axis “L”) of the patient supporting surface of surgical table 10 to provide better clearance and/or approach for performing ACDF (e.g., to facilitate use of a microscope during the ACDF). Adjustment mechanism 16 may additionally or alternatively enable forward and rear tilting of the patient supporting surface of surgical table 10 (e.g., between neutral, Trendelenburg, and reverse Trendelenburg positions). Height adjustment of surgical table 10 using adjustment mechanism 16 is also contemplated, e.g., to reposition surgical table 10 based upon the height of the surgeon “S,” based upon whether the surgeon “S” prefers a standing or sitting approach, etc. Tilt and/or height adjustment of surgical table 10 using adjustment mechanism 16 may be powered, e.g., via one or more motors of adjustment mechanism 16.

Referring to FIG. 5B, another surgical table 500 provided in accordance with the present disclosure and configured to facilitate positioning the patient “P” is shown. Surgical table 500 includes a patient support surface 510 operably mounted between first and second supports 522, 524 of a table frame 520 to enable airplaning, forward and rear tilting, and/or height adjustment of the patient support surface 510 relative to first and second supports 522, 524 of table frame 520. Adjustment of surgical table 500 may be powered, e.g., via one or more motors, and, in such aspects, may be controlled by a processor to enable precise placement and/or movement of patient support surface 510. Table frame 520 of surgical table 500 may further include a manipulation apparatus mount 526 configured to enable mounting of a manipulation apparatus 750 thereto, as detailed below. In aspects, manipulation apparatus mount 526 may electromechanically mount to manipulation apparatus 750 to enable powered and controlled manipulation of manipulation apparatus 750.

Continuing with reference to FIG. 5B, in aspects, table frame 520 of surgical table 500 further includes a support extension 530 engaged to patient support surface 510 and extending therefrom to support a portion of the patient “P” that extends beyond patient support surface 510, e.g., an arm of the patient “P.” Additional cushioning 540 may be utilized to support the patient “P” on surgical table 500 such as, for example, supporting the head of the patient “P” between manipulation apparatus 750 and patient support surface 510. To the extent consistent, surgical table 500 may include any of the features of surgical table 10 (FIG. 5A), and vice versa.

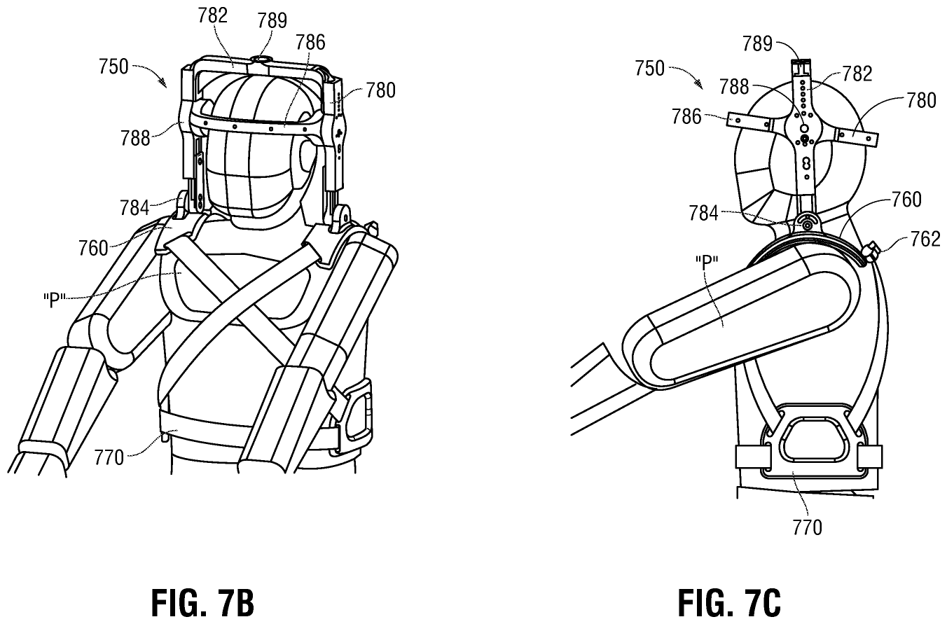

With reference to FIG. 7A, in aspects, in order to facilitate positioning the head and neck of the patient “P,” such as to achieve the positions detailed above or otherwise herein, a manipulation apparatus 700 may be utilized. Manipulation apparatus 700 includes a base frame 710 configured to mount to the body of the patient “P” and a movable frame 720 configured to attach to the head of the patient “P.” Movable frame 720 includes one or more first (e.g., flexion/extension) joints 722 configured to enable pivoting of movable frame 720 relative to base frame 710 to thereby enable movement of the head of the patient “P” between a neutral position, a flexion position, and an extension position. Movable frame 720 may additionally or alternatively include one or more second (e.g., side-to-side) joints 724 configured to enable pivoting of movable frame 720 relative to base frame 710 to thereby enable movement of the head of the patient “P” laterally to achieve a desired position.

As detailed above, manipulation apparatus 700 enables controlled movement of the head and neck of the patient “P” to a desired position. Manipulation apparatus 700 is further configured to selectively maintain, or lock, the position of the head and neck of the patient “P” once the desired position is achieved. In aspects, manipulation apparatus 700 is manually actuated, e.g., via knobs or direct movement of movable frame 720 relative to base frame 710, and movable frame 720 is manually lockable relative to base frame 710. In other aspects, manipulation apparatus 700 is motorized to enable powered positioning and retention of the head and neck of the patient “P” in a desired position.

With reference to FIGS. 7B and 7C, another manipulation apparatus 750 is provided in accordance with the present disclosure. Manipulation apparatus 750 includes a shoulder frame 760 (e.g., shoulder supports, which may include pads for patient comfort) configured to sit on the shoulders of the patient “P,” a securement device 770 (e.g., straps) configured to secure shoulder frame 760 relative to the patient “P,” and a movable frame 780 configured to attach to the head of the patient “P.” Movable frame 780 may be configured as a Mayfield frame mounted to the skull of the patient “P.” Movable frame 780, more specifically, includes a first frame 782 pivotably connected to shoulder frame 760 on either side of the head of the patient “P” about first joints 784. First frame 782 extends over the head of the patient “P.” Movable frame 780 further includes a second frame 786 pivotably connected to first frame 782 on either side of the head of the patient “P” via second joints 788. Second frame 788 extends circumferentially about the head of the patient “P.”

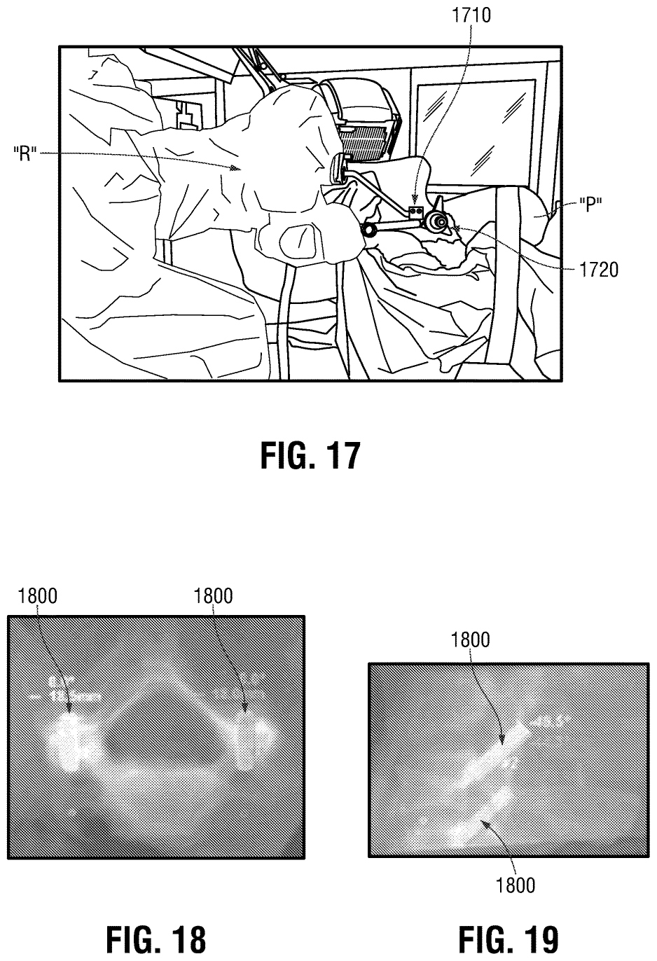

Second frame 788 includes a mounting portion 789 configured to enable mounting of manipulation apparatus 750 to a surgical table, e.g., to manipulation apparatus mount 526 of surgical table 500 (see FIG. 5B). Additionally or alternatively, shoulder frame 760 may include a robotic mount 762 configured to enable mounting of a robotic surgical device “R” (see FIG. 17) thereto, thus defining a support location and/or point of reference fixed relative to the patient “P.” To the extent consistent, manipulation apparatus 750 may include any of the features of manipulation apparatus 700 (FIG. 7A), and vice versa.

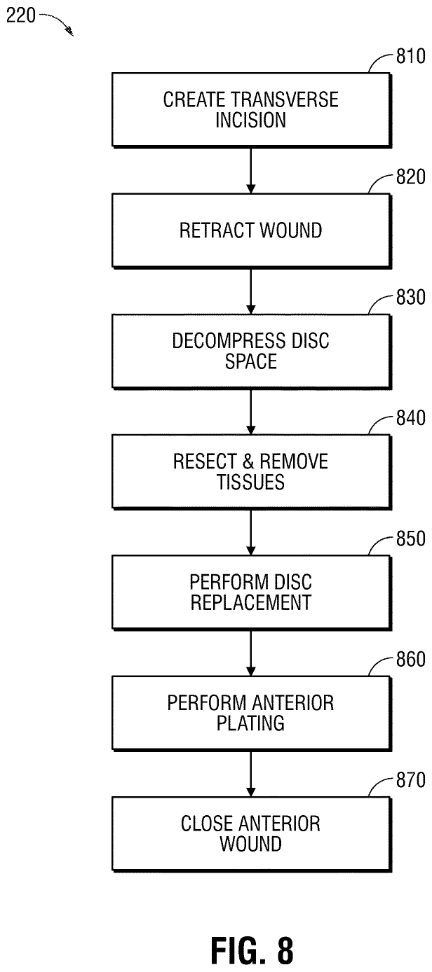

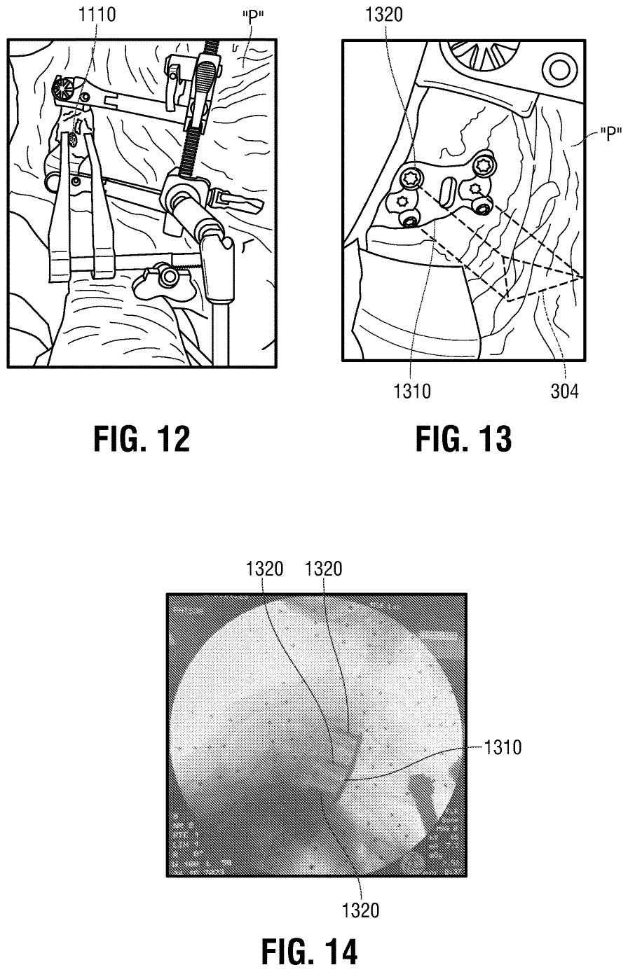

Referring to FIGS. 8-14, performing ACDF 220 (see also FIG. 2) includes creating an incision 810 (e.g., a transverse incision or a longitudinal incision along the neck), retracting the wound 820, decompressing the disc space 830, resecting and removing tissues 840, performing disc replacement 850, performing anterior plating 860, and closing the anterior wound 870. The ACDF may be performed with the surgeon “S” (FIGS. 1 and 3) in a standing or sitting position. In aspects, the surgeon “S” (FIGS. 1 and 3) may utilize a microscope to facilitate visualization.

With respect to creating the transverse incision 810, a standard transverse incision may be made. In addition, the musculature and other tissues are bisected as necessary to expose the vertebral bodies to be fused.

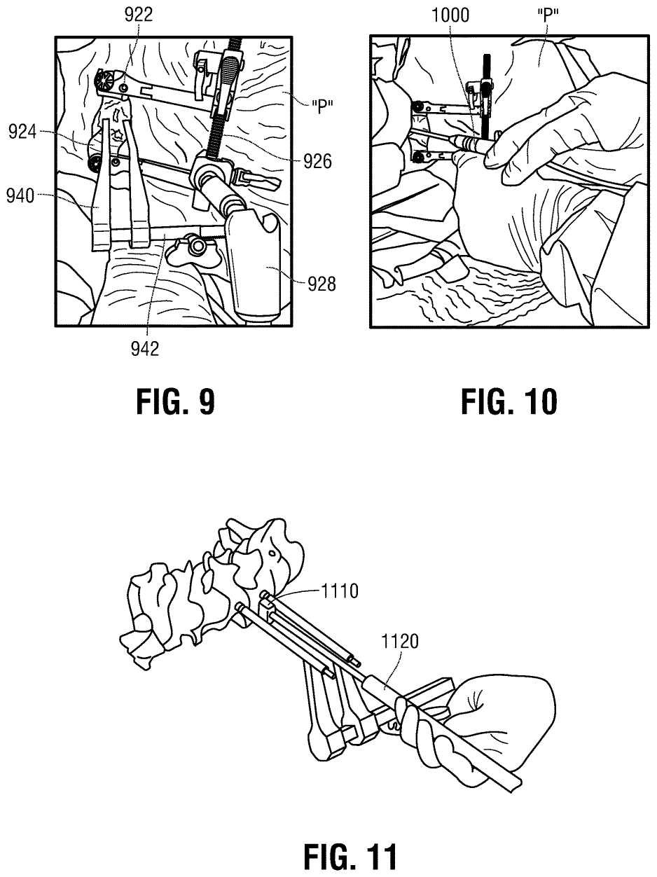

Next, the wound is retracted 820. Referring to FIGS. 8 and 9, wound retraction may be accomplished using suitable retractor blades 922, 924 oriented vertically (vertically relative to the surgeon “S” (FIGS. 1 and 3); laterally across the patient “P”) such one retractor blade is a top retractor blade 922 and the other retractor blade is a bottom retractor blade 924. This positioning facilitates visibility of the surgical site. Wound retraction, more specifically, may be performed by inserting the top and bottom retractor blades 922, 924 and connecting a retractor rack 926 to the retractor blade 922, 924 to enable movement of the retractor blades 922, 924 to achieve the necessary retraction. A table support arm 928 secured to surgical table 10 (FIG. 3), or other suitable support, e.g., tape, may be utilized to maintain the position of the retractor components 922, 924, 926 relative to the patient “P,” e.g., to counteract gravity. In aspects, a surgical assistant positioned on the posterior side of the patient “P” (e.g., on an opposite side of the surgical table 10 as compared to the surgeon “S” (see FIG. 3)) may facilitate insertion, positioning, and retention of the retractor components 922, 924, 926, e.g., by reaching over the patient “P.”

Continuing with reference to FIGS. 8 and 9, decompressing the disc space 830 may include utilizing a distractor 940 to expand the disc space(s) of interest. Where a distractor 940 is utilized, a rack 942 associated with the distractor 940 may be positioned below the wound (e.g., closer to the surgical table 10 (FIG. 3)) to maintain visibility. A microscope (not shown) may be positioned, e.g., on the surgical table 10 (FIG. 3), at this point, prior thereto, or at any other suitable point, to facilitate visualization of the discs and disc space(s) of interest.

With reference to FIGS. 8 and 10, resection and removal of tissue 840 includes resecting the anterior longitudinal ligament, discectomy, decompression of neural elements, and/or removal of anterior osteophytes. This may be accomplished with any suitable surgical tools 1000 (e.g., hand-operated tools operated by the surgeon “S” (FIG. 3)) and, in particular, those surgical tools 1000 that enable the resection and removal of tissue 840 without interference from or collision with the downward arm of the patient “P.” For example, tools 1000 having pencil grips, rotatable grips, or other grips without significant downwardly protruding portions may be desirable.

Referring to FIGS. 8, 11, and 12, performing disc replacement 850 may initially include distraction and trialing to size the disc space. Once the disc space is sized, the disc replacement 1110, e.g., bone graft, implant, or other suitable replacement, is inserted into position utilizing any suitable tools 1120 (e.g., hand-operated tools operated by the surgeon “S” (FIG. 3)). During disc replacement 850, additional support (e.g., using one or more supports 12 (FIGS. 3 and 5A)) may be provided on the posterior side of the neck of the patient “P” to counteract the forces associated with trialing and impaction of the disc replacement, thereby avoiding movement of the patient “P.” In aspects, a support 12 (FIGS. 3 and 5A) is added, repositioned, and/or adjusted to provide the additional support on the posterior side of the patient “P.” The additional support may be removed at the completion of the disc replacement 850, at the completion of the anterior plating 860, or at any other suitable point. In aspects where a graft or other implant is utilized, the graft or implant may be inserted through a tube, through a screw having a hollow interior, and/or through a delivery instrument, such as a malleable delivery tool enabling access, e.g., through a tortuous path, to the disc space for insertion.

Turning to FIGS. 13 and 14, in conjunction with FIG. 8, once disc replacement 850 is completed, anterior plating 860 is performed wherein an anterior surgical plate 1310 is attached to the disc replacement 1110 and the vertebrae on either side of the disc replacement 1110 using suitable surgical screws 1320. Given the lateral positioning of the patient “P” and, thus, gravity considerations, proper alignment of the anterior surgical plate 1310 relative to the patient “P” (e.g., the spinal anatomy) during anterior plating 860 is maintained. Anterior plating 860 may be performed with, for example, hand-operated tools operated by the surgeon “S” (FIG. 3).

With particular reference to FIG. 13, in aspects, once anterior plating 860 is completed, a navigation component including an emitter array 304 is mounted on anterior surgical plate 1310. Since anterior surgical plate 1310 is fixed to the spine of the patient “P,” securing the emitter array 304 to the anterior surgical plate 1310 (and/or surgical screws 1320) provides a fixed point of reference relative to the spine of the patient “P” to facilitate surgical planning and/or surgical navigation, e.g., for performing transfacet screw fixation 230 (FIG. 2), as detailed above. In such aspects, surgical plate 1310 (and/or surgical screws 1320) may include suitable features such as, for example, threaded recesses, key holes, tabs, and/or other mechanical features to enable releasable securement of the emitter array 304 relative to anterior surgical plate 1310. In other aspects, other reference navigation components are utilized.

Referring again to FIG. 8, to complete the ACDF procedure 220, the anterior wound is closed, as indicated at 870. Closure of the anterior wound 870 may include suturing.

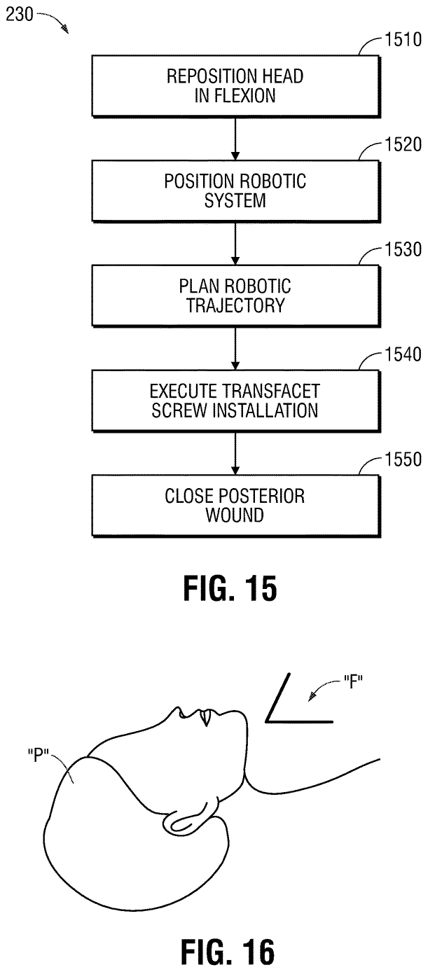

With reference to FIGS. 15-20E, in conjunction with FIG. 2, after completion of the ACDF procedure 220, the single position 360 degree cervical fusion method 200 next includes performing transfacet screw fixation (as indicated at 230). Since the single position 360 degree cervical fusion method 200 involves maintaining the patient “P” in a lateral position, turning or substantial repositioning of the patient is not required. However, and referring to FIGS. 15 and 16, performing transfacet screw fixation 230 may initially include, as indicated at 1510, repositioning the head of the patient “P” (from the extension position “E” (FIG. 6), which was used to facilitate performing the ACDF (FIGS. 2 and 8)) such that the head of the patient “P” is in flexion “F” or otherwise positioned so as to facilitate posterior access. For example, positioning the head of the patient “P” in flexion “F” may facilitate posterior access by helping to ensure that the skull does not interfere with the transfacet screw insertion, detailed below.

The above-noted repositioning may be performed using manipulation apparatus 700 (FIG. 7A), manually, or in any other suitable manner. In aspects, the patient “P” is re-secured to the surgical table 10 (FIG. 3) once the desired position for the transfacet screw fixation 230 has been achieved. Further, if necessary, surgical table 10 (FIG. 3) is tilted and/or height adjusted (e.g., as detailed above) to achieve a desired position for the transfacet screw fixation 230.

Turning to FIGS. 15 and 17, the transfacet screw fixation 230 next includes positioning the robotic system, as indicated at 1520. With regard to positioning the robotic system 1520, the robotic system “R” is positioned on or otherwise in relation to the surgical table 10 on the posterior side of the patient “P.” Further, the robotic system “R” may be attached to the spine of the patient “P,” e.g., at T1-C7, using a spinous process clamp 1710 and coupler 1720. In aspects, at least a portion of the spinous process clamp 1710 and/or coupler 1720 is formed from a radiolucent material to enable visualization and/or identification thereof on an x-ray image. Thus, either or both of these components 1710, 1720 may be utilized as a reference point during surgical planning and/or navigation. Imaging scanning and registration may also be performed once the robotic system “R” is positioned as detailed above and/or to facilitate the positioning.

With reference to FIGS. 15, 18, and 19, the transfacet screw fixation 230 next includes planning the robotic trajectory, as indicated at 1530. Robotic trajectory planning 1530 includes adjusting the segment lines to ensure clear view of the target facet joints. The trajectory is selected to leave sufficient inferior and superior articular process above and below the screws and to perforate the facet. Further, the trajectory and/or delivery depth are selected to aim for the bottom of the disc space, avoid penetration of, or at least overpenetration of, the anterior or ventral surface of the superior articular process, and attempt to maintain substantially perpendicular orientation relative to the facet surface. Exemplary trajectories 1800 in axial view and lateral view, respectively, are illustrated in FIGS. 18 and 19.

With respect to robotic trajectory planning 1530, more specifically, the trajectories 1800 are determined, in an axial view (see FIG. 18), such that the screws are positioned straight on or slightly angled to ensure adequate bone purchase and to enable maximization of the diameter and length of screws utilized. In the lateral view (see FIG. 19), the trajectories 1800 are made substantially perpendicular to the facet joints to ensure robust fastening of the joint. High angles are to be avoided as they may cause reachability issues for the arm of the surgical robot “R” (FIG. 17). Further, alignment of the trajectories at the skin level can be accomplished to minimize incision size and promote cosmesis.

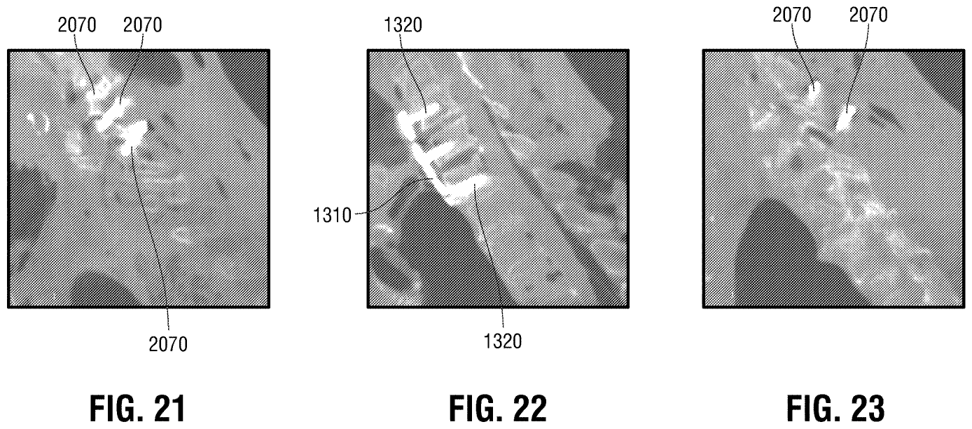

Turning to FIGS. 20A-20E, with the robotic trajectory planned at 1530, the robotic transfacet screw installation may be executed, as indicated at 1540. More specifically, the arm guide 2010 is initially sent to the pre-planned trajectory (see FIG. 20A); a scalpel 2020 is inserted through the arm guide 2010 to make an incision through the skin “K” to bone level “B” (see FIG. 20B); a cannula 2030 and dilator 2040 are inserted through the arm guide 2010 to assess the contour of the bone and trajectory (see FIG. 20C); the dilator 2040 is withdrawn from the cannula 2030, leaving the cannula 2030 in position; a surgical drill 2050 is inserted through the cannula 2030, accelerated, and advanced into bone utilizing navigation (e.g., via navigation component 3052, or using another depth control method) to perforate the facet (se FIG. 10D); the surgical drill 2050 is then removed from the cannula 2030 (leaving the cannula in position); and a screw driver 2060, including a screw 2070 (FIGS. 21 and 23) retained thereon, is inserted through the cannula 2030 to advance the screw 2070 (FIGS. 21 and 23) to the desired (e.g., pre-planned) depth (see FIG. 20E). As noted above, some or all of the above may be performed robotically and/or using one or more robotically guided tools. Alternatively, some or all of the above may be performed with handheld instrumentation.

The above is repeated as necessary to install all of the screws 2070 associated with the transfacet screw installation 1540. Referring back to FIG. 15, upon completion of the transfacet screw installation 1540, the posterior wound is closed, as indicated at 1550, e.g., using sutures.

FIGS. 21-23 illustrate results of the above-detailed single position 360 degree cervical fusion method 200 (FIG. 2) in accordance with the present disclosure. More specifically, in conjunction with FIG. 2, FIG. 21 illustrates the installed left side screws 2070 of the transfacet fixation 230, FIG. 22 illustrates the screws 1320 and plate 1310 of the ACDF 220, and FIG. 23 illustrates the installed right side screws 2070 of the transfacet fixation 230.

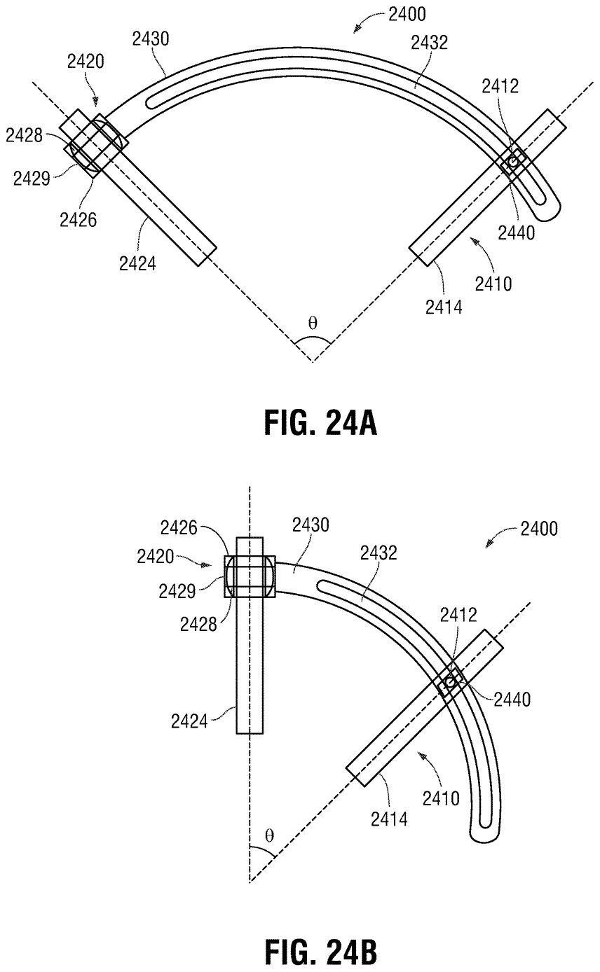

Referring to FIGS. 24A and 24B, a coupling assembly 2400 provided in accordance with the present disclosure is shown generally identified by reference numeral 2400. Coupling assembly 2400 includes first and second portals 2410, 2420 coupled to one another by a coupling arm 2430. More specifically, second portal 2420 is engaged with coupling arm 2430 while first portal 2410 is coupled with coupling arm 2430 by a pin-slot engagement including a transverse pin 2412 extending from first portal 2410 received within an arcuate slot 2432 defined within coupling arm 2430, although other coupling mechanism are also contemplated. In aspects, coupling arm 2430 defines a bifurcated configuration including first and second spaced-apart arm portions configured to receive first portal 2410 therebetween, wherein each arm portion defines a slot 2432 configured to receive a pin 2412 or a portion of a pin 2412.

Transverse pin(s) 2412 is confined by arcuate slot(s) 2432 in two perpendicular planes to thereby fix the trajectories of first and second portals 2410, 2420 relative to one another in two perpendicular planes. However, transverse pin(s) 2412 are configured to slide along arcuate slot(s) 2432 to thereby move first portal 2410 relative to second portal 2420 about an arc defined by arcuate slot(s) 2432 and within a third perpendicular plane. The arc defined by arcuate slot 2432 may be radiused about a center disposed on a longitudinal axis of second portal 2420 such that first portal 2410 is movable relative to second portal 2420 to vary a trajectory angle θ defined between longitudinal axes (e.g., the trajectories) of first and second portals 2410, 2420.

A locking mechanism 2440, e.g., a set screw, mechanical latch, etc., may be provided to selectively lock the position of first portal 2410 along coupling arm 2430, thereby locking the trajectories of first and second portals 2410, 2420 relative to one another in the third perpendicular plane. In aspects, locking mechanism 2440 locks the trajectories at a fixed trajectory angle θ; in other aspects, locking mechanism 2440 locks the trajectories within a fixed trajectory range around the trajectory angle θ, e.g., plus or minus 2 degrees, plus or minus 5 degrees, etc.

Either or both of first and second portals 2410, 2420 may define elongate cannulae 2414, 2424 (as shown) configured to receive surgical tools, devices, and/or materials therethrough, may define collars configured to receive surgical tools, devices, and/or materials therethrough, and/oy may define any other suitable configurations for receiving or otherwise coupling to surgical tools, devices, and/or materials to guide the surgical tools, devices, and/or materials along defined trajectories to a surgical site.

In aspects, second portal 2420 includes a collar 2426 configured to slidably receive cannula 2424, thereby enabling longitudinal adjustment of cannula 2424 along its longitudinal axis and relative to first portal 2410 and coupling arm 2430. In such aspects, a locking mechanism 2428 e.g., a set screw, mechanical latch, etc., is provided to selectively lock cannula 2424 relative to collar 2426 and, thus, relative to coupling arm 2430. As an alternative or in addition to enabling sliding of cannula 2424, collar 2426 may include a joint 2429 configured to permit monoaxial, polyaxial, or unlimited pivoting of cannula 2424 relative to collar 2426, thus enabling further repositioning of second portal 2420 relative to first portal 2410. Locking mechanism 2428 e.g., a set screw, mechanical latch, etc., or a separate locking mechanism, in aspects, may be provided to lock joint 2429, thereby fixing the position of cannula 2424 relative to coupling arm 2430.

With additional reference to FIG. 25, coupling assembly 2400 may be utilized to guide surgical tools, devices, and/or materials to a surgical site, for example, posterior portions of a patient's spinal anatomy, e.g., a facet joint “FJ” and/or posterior bone surface. More specifically, coupling assembly 2400 may be utilized to facilitate single position 360 degree cervical fusion in accordance with the present disclosure and as detailed hereinabove.

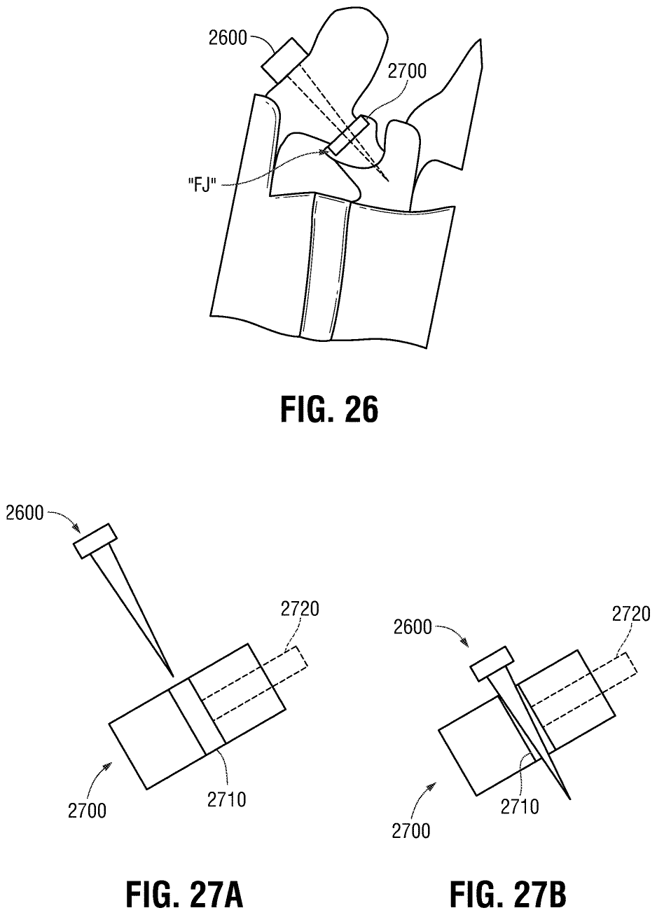

In aspects, first portal 2410 is configured to receive a first surgical instrument 2510 to guide the first surgical instrument to a facet joint “FJ.” The first surgical instrument 2510 may be utilized to decorticate the joint “FJ” and/or the first surgical instrument 2510 or another surgical instrument may be inserted through first portal 2410 to deliver a component 2700 (FIG. 26) such as a device or material, e.g., a graft material, a mechanical cage, etc., to the joint “FJ.” The first (and/or another) surgical instrument 2510 may be a robotic surgical instrument connected to a robotic arm of a surgical robotic system, although handheld instruments, table-mounted instruments, and/or other instruments are also contemplated.

A second surgical instrument 2520 may be inserted through second portal 2420 such that, due to the coupling of first and second portals 2410, 2420, the angular orientations of first and second instruments 2510, 2520 relative to one another are fixed (or constrained), e.g., according to the positions of first and second portals 2410, 2420. Second surgical instrument 2520 may be a robotic surgical instrument connected to a robotic arm of a surgical robotic system, although handheld instruments, table-mounted instruments, and/or other instruments are also contemplated.

Referring also to FIG. 26, second surgical instrument 2520 may be utilized, for example, to introduce a transfacet screw 2600 through the facet joint “FJ” to immobilize the facet joint “FJ.” In aspects, the transfacet screw 2600 is configured to engage the component 2700 previously delivered to the joint “FJ.” Coupling assembly 2400 facilitates guidance of second surgical instrument 2520 for delivery of the transfacet screw 2600 through the facet joint according to a surgical plan. More specifically, coupling assembly 2400 may be adjusted to fix the trajectory of second portal 2420 (and, thus, second surgical instrument 2520 and transfacet screw 2600) at 90 degrees in the coronal plane relative to the trajectory of first portal 2410 (and, thus, first surgical instrument 2510) such that alignment of first surgical instrument 2510 at the joint space of facet joint “FJ” aligns the transfacet screw 2600 perpendicular to the joint space of facet joint “FJ.” However, other trajectory angles O are also contemplated.

In aspects, the trajectory angle O of second portal 2420 relative to first portal 2410 may be determined using one or more navigated and/or image-guided tools, e.g., of surgical navigation system 306 (FIG. 1). For example, first surgical instrument 2510 may be inserted through first portal 2410 and positioned at the target site. A navigated and/or image-guided device may then be inserted through second portal 2420 or otherwise positioned to determine a target trajectory of second portal 2420. Second portal 2420 may then be adjusted relative to first portal 2410 and locked in position (or within a range of positions) such that, upon insertion of second surgical instrument 2520 through second portal 2420, second portal 2420 guides second surgical instrument 2520 to the target location along the determined trajectory.

Referring to FIGS. 27A and 27B, in aspects, the component 2700 disposed within the joint space, e.g., the graft material, mechanical cage, etc., may be configured to receive the transfacet screw 2600 (and/or other bone screw) and retain the screw 2600 in position once it passes through the component 2700. For example, the component 2700 may include a passage 2710 configured to expand and engage the screw 2600 upon insertion of screw 2600 therethrough. Alternatively, the component 2700 may be configured to collapse passage 2710 to engage the screw 2600 after insertion of screw 2600 therethrough. Other suitable lock mechanism configured to retain the screw 2600 are also contemplated. Regardless of the particular lock mechanism, locking may be passive, e.g., wherein the component 2700 is configured to engage the screw 2600 in response to insertion of the screw 2600, or active, e.g., wherein the component 2700 is configured to engage the screw 2600 upon actuation of the lock mechanism such as, for example by an actuator 2720 (accessible through first portal 2410, for example (see FIGS. 24A-25)).

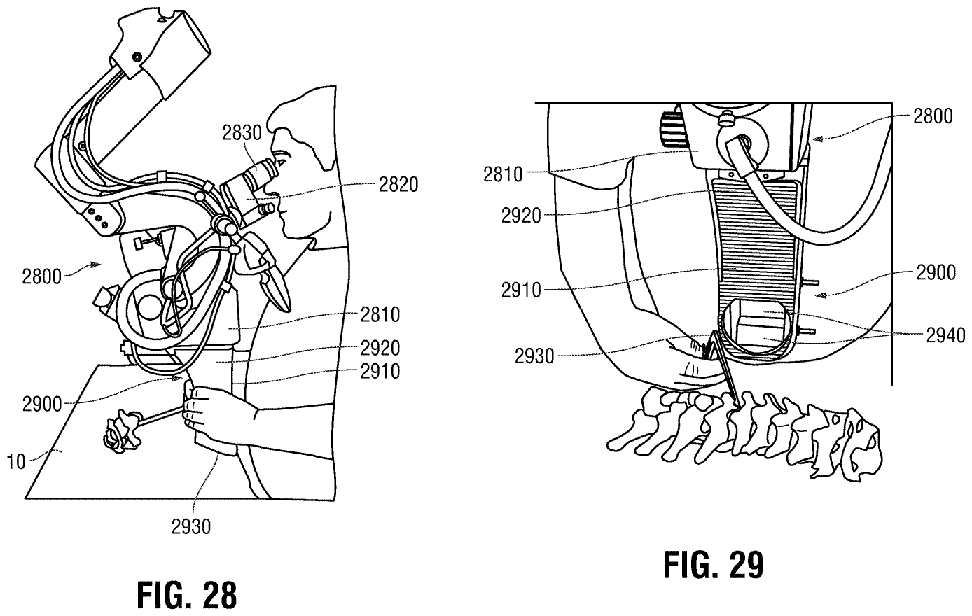

Turning to FIGS. 28 and 29, as noted above, a surgical microscope 2800 may be utilized to facilitate visualization during an ACDF procedure. A surgical microscope 2800 allows for ergonomic positioning of the surgeon relative to the patient when the surgical site is oriented upwardly away from the floor, e.g., during an ACDF procedure with the patient in the supine position. However, when the patient is in a lateral position, the microscope 2800 may need to be repositioned such that the direction of view is substantially parallel relative to the floor. In this configuration, the length of the microscope 2800 requires the surgeon to stretch their arms out (horizontally) to reach the surgical site while viewing the surgical site through microscope 2800, which is not an ergonomic position.

Surgical periscope 2900 provided in accordance with the present disclosure improves the ergonomics for the surgeon by repositioning and redirecting the direction of view of the microscope 2800 by a suitable angle (fixed or adjustable) such as, for example 90 degrees, although other suitable angles as well as angle ranges, e.g., from about 45 degrees to about 135 degrees, are also contemplated.

Surgical periscope 2900 includes a housing 2910 configured to releasably engage objective end 2810 of microscope 2800 at a first end open end 2920 of housing 2910 of surgical periscope 2900 and extending from first open end 2920 to a second open end 2930 of housing 2910 of surgical periscope 2900. Second open end 2930 is disposed at a fixed or adjustable angle relative to first open end 2920, thus defining the angle of the change in viewing direction of microscope 2800 when surgical periscope 2900 is utilized. Housing 2910 may define a transverse width (in a direction along the surgical table 10 and/or across the surgical table 10) that is equal to or less than the same width of the housing of microscope 2800 at objective end 2810 thereof, thus providing sufficient space around surgical periscope 2900 for the surgeon to maneuver their arms to perform a surgical task at the surgical site.

Within housing 2910, surgical periscope 2900 includes one or more optical elements 2940 such as, for example, mirrors, prisms, lenses, etc., and/or electronic optical elements such as cameras, image sensors, etc., configured to relay the field of view in the viewing direction of second open end 2930 of housing 2910 of surgical periscope 2900 to first open end 2920 of housing 2910 of surgical periscope 2900 and, thus, objective end 2810 of microscope 2800 such that the surgeon may view, through eye pieces 2830 at viewing end 2820 of microscope 2800, the field of view in the viewing direction of second open end 2930 of housing 2910 of surgical periscope 2900 (at any suitable magnification of microscope 2800). Thus, the surgeon can stand closer to the surgical site, reducing or eliminating the need for the surgeon to stretch out towards the surgical site, and enabling the surgeon to stand upright in an ergonomic position.

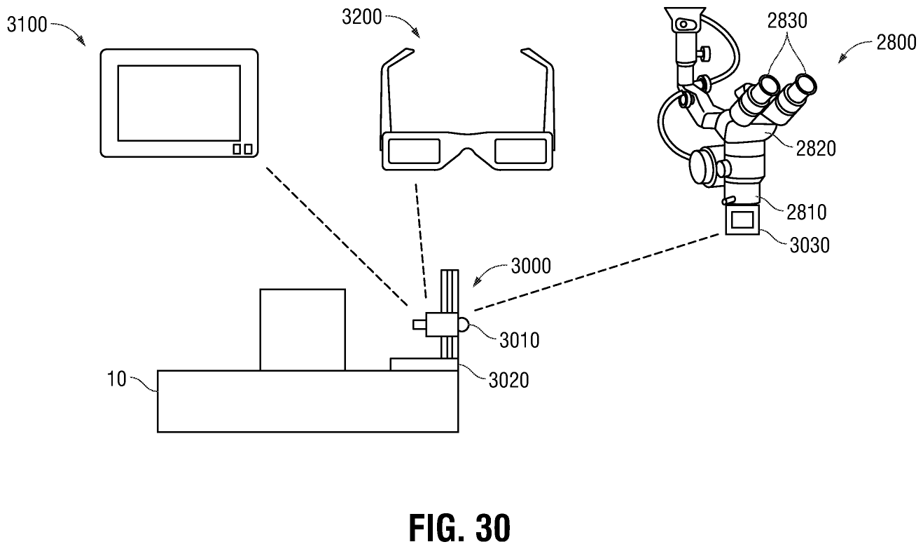

Turning to FIG. 30, as an alternative to physically connecting surgical periscope 2900 to microscope 2800 (FIGS. 28 and 29), a surgical periscope 3000 may be provided to wirelessly connect to microscope 2800 and/or another suitable device such as, for example, a display screen 3100, display glasses 3200, etc. Surgical periscope 3000 includes an image capture portion 3010, which may be configured for positioning on surgical table 10. In aspects, image capture portion 3010 is movably mounted on a base 3020 configured to enable adjustment of a height and/or angle of the viewing direction of image capture portion 3010. Image capture portion 3010 includes one or more cameras, image sensors, etc. configured to capture an image, e.g., video, of the surgical site. Image capture portion 3010 further includes a wireless interface (not shown) configured to wirelessly transmit, via Wifi, Bluetooth, Infrared (IR) communication, any/or any other wireless transmission method, the captured image, e.g., video, to a receiver portion 3030 of surgical periscope 3000 and/or directly to another device, e.g., display screen 3100, display glasses 3200, etc. Receiver portion 3030, where provided, is configured to reproduce the image captured by image capture portion 3010 at objective end 2810 of microscope 2800 such that the surgeon may view, through eye pieces 2830 at viewing end 2820 of microscope 2800, the captured image. Receiver portion 3030 may be configured to releasably engage microscope 2800 at objective end 2810 thereof.

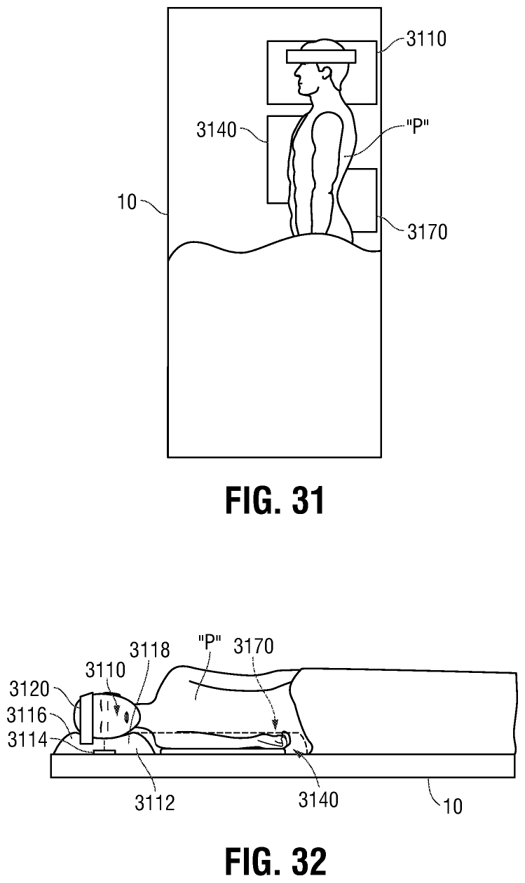

Referring to FIGS. 31-34, similarly as detailed above with reference to FIGS. 3-6, the patient “P” may be supported on the surgical table 10 in the lateral position to facilitate performing one or more surgical tasks, e.g., to facilitate performing ACDF. In order to support the patient “P” in the lateral position, a plurality of supports 3110, 3140, 3170 may be provided.

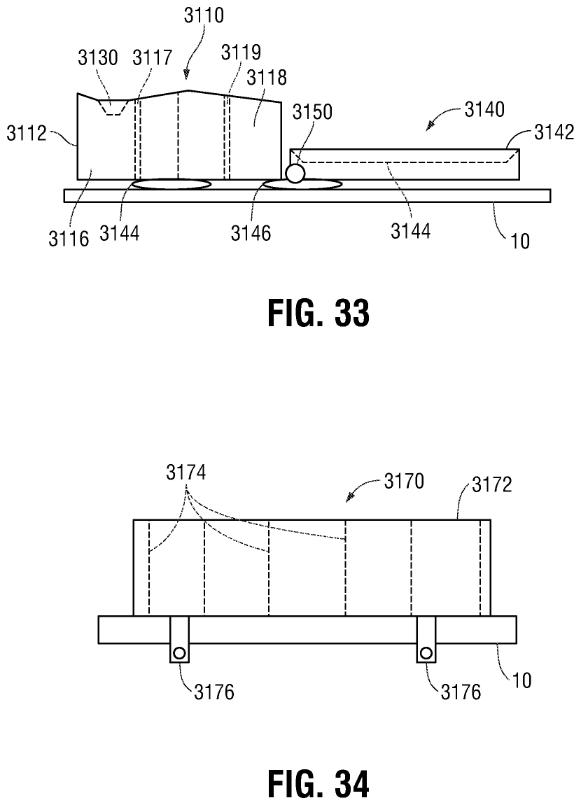

Support 3110, as shown in FIGS. 32 and 33 includes a body 3112 rotatably coupled to a mount 3114 configured for positioning on surgical table 10 such that body 3112 is rotatable relative to mount 3114 and surgical table 10 about an axis perpendicular to the patient support surface defined by surgical table 10. Thus, the head and neck of the patient “P” can be rotated anteriorly or posteriorly relative to the torso of the patient “P.” Body 3112 of support 3110 is configured to support at least a portion of a head and neck of the patient “P” and may include a plurality of sections 3116, 3118 arranged longitudinally relative to the longitudinal axis of the patient “P” such that the first section 3116 supports the head (or more of the head) of the patient “P” while the second section 3118 supported the neck (or more of the neck) of the patient “P.” Although two sections 3116, 3118 are detailed, it is also contemplated that more than two sections 3116, 3118 can be provided. Body 3112 may be formed from or include a cushioned material to facilitate comfort of the patient, although other configurations are also contemplated.

First and second sections 3116, 3118 are independently adjustable, e.g., via adjustment mechanisms 3117, 3119, to independently vary the heights of first and second sections 3116, 3118, thus enabling selective positioning of the head and/or neck of the patient “P” relative to one another and/or the torso of the patient “P.” Adjustment mechanisms 3117, 3119 may include ratchets, lead screws, telescoping mechanisms, inflatable bladders, motors, and/or any other suitable mechanisms enabling selective adjustment of the heights of first and second sections 3116, 3118, respectively.

In aspects, adjustment mechanisms 3117, 3119 are automatically adjustable to set first and second sections 3116, 3118 to selected heights such as, for example, via computer-controlled motors of adjustment mechanisms 3117, 3119. More specifically, different heights of first and second sections 3116, 3118 may be required during the course of a surgical procedure to reposition the patient “P” to facilitate performing one or more tasks of the surgical procedure. In such aspects, a user input to the computer controlling the motors of adjustment mechanisms 3117, 3119, e.g., of the step or status of the procedure to be performed, enables automatic adjustment of first and second sections 3116, 3118 to the selected heights for that portion of the procedure. As another example, different heights of first and second sections 3116, 3118 may be required for different patient anatomies and, thus, a user input to the computer controlling the motors of adjustment mechanisms 3117, 3119, e.g., of the patient or anatomy of the patient, enables automatic adjustment of first and second sections 3116, 3118 to the appropriate height.

Support 3110 may further include a strap 3120 configured to secure the head of the patient “P” to support 3110. Strap 3120 may attach to opposing sides of body 3112 at opposing sides thereof, or may be provided in any other suitable manner. Additionally or alternatively, first section 3116 of body 3112 includes an ear hole 3130 formed as an aperture, cut-out, recess, and/or in any other suitable manner such that ear hole 3130 receives at least a portion of the ear of the patient “P” to more comfortably and stably support the head of the patient “P” on support 3110.

With continued reference to FIGS. 32 and 33, support 3140 includes an elongate body 3142 defining an elongate recess 3144 configured to receive at least a portion of an arm of the patient “P,” e.g., an upper arm of the patient “P.” Support 3140 further includes a base 3146 and a pivot 3150 pivotably coupling base 3146 and body 3142 with one another. Base 3146 is configured for positioning on or engagement with surgical table 10 such that body 3142 is pivotable about pivot 3150 and relative to base 3146 and surgical table 10 about an axis perpendicular to the patient support surface defined by surgical table 10. In this manner, the lower arm of the patient “P” can be moved towards or away from the torso of the patient “P” as needed to provide operating room, provide clearance, enable visualization, etc. to facilitate performing the surgical procedure.

Referring to FIGS. 31, 32, and 34, support 3170 is configured to support a posterior side of the patient “P.” In aspects, support 3170 includes a body 3172 having one or more reinforcement members 3174, e.g., of stronger material than the material forming body 3172, thus providing additional structural support for supporting the patient “P.” Additionally, support 3170 may be secured to the surgical table 10, e.g., via clamps 3176 or in any other suitable manner. The securement of support 3170 to the surgical table 10 and/or the additional structural support provide to support 3170 ensures proper support of the posterior side of the patient “P,” and maintains the patient “P” in position on the surgical table 10 such as, for example when the surgical table 10 is airplaned with the posterior side of the patient “P” disposed below the anterior side of the patient “P.”

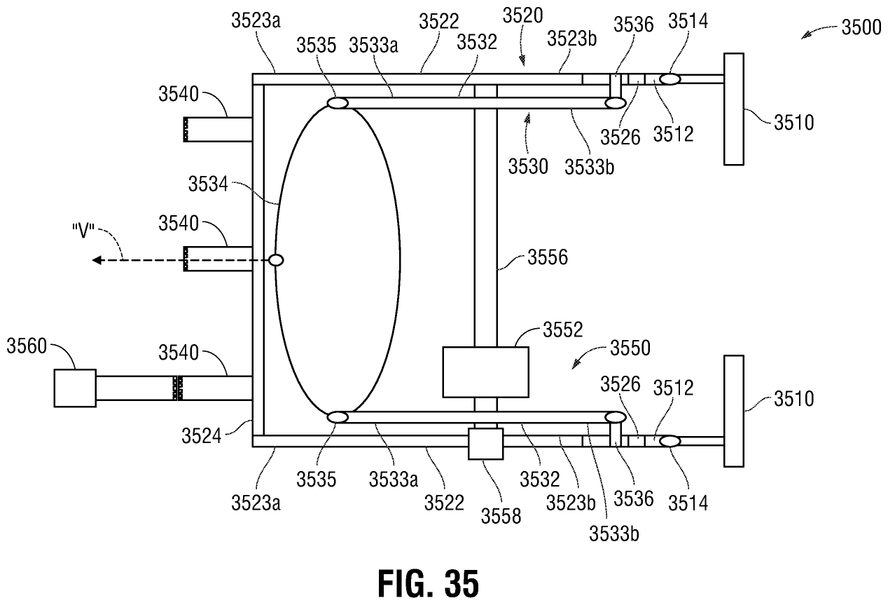

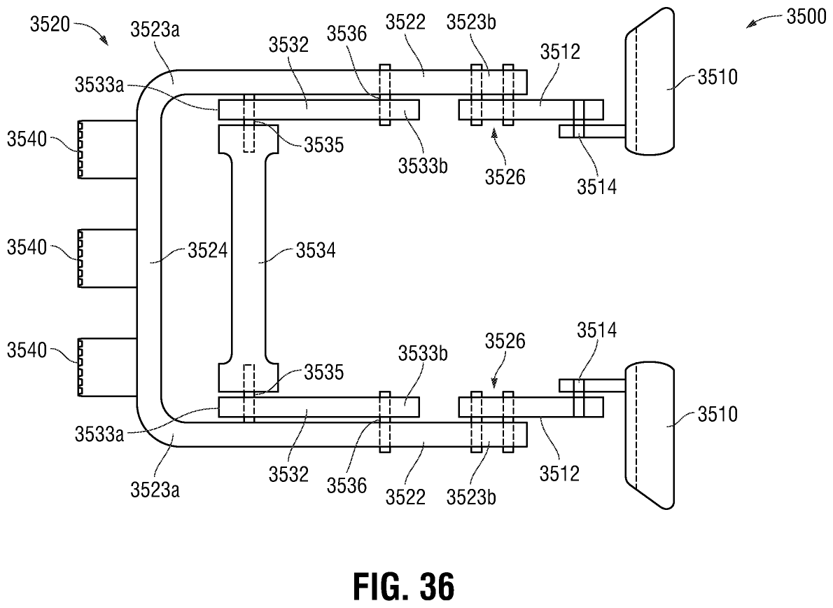

Referring to FIGS. 35-37B, as noted above, manipulation apparatus 700, 750 (FIGS. 7A-7C) may be provided to facilitate positioning the head and neck of the patient “P,” (FIGS. 7A-7C) such as to achieve positions to facilitate performing the ACDF as detailed above. Manipulation apparatus 3500, detailed hereinbelow, may include any of the features of manipulation apparatus 700, 750 (FIGS. 7A-7C), except as explicitly contradicted.

Manipulation apparatus 3500 includes one or more base supports 3510 configured to engage the torso of a patient, an outer frame 3520 coupled to and extending from base support(s) 3510 to surround the head and neck of the patient, an inner frame 3530 coupled to and disposed within outer frame 3520, one or more mounting extensions 3540, and an adjustable bolster assembly 3550. Manipulation apparatus 3500 is detailed below with outer frame 3520 as the fixed reference; however, it is also contemplated that other components of manipulation apparatus 3500 may be fixed and/or that all components of manipulation apparatus 3500 may be movable, e.g., such as where manipulation apparatus 3500 is detached from the support and worn by the patient to facilitate recovery from surgery and/or for other purposes.

The one or more base supports 3510 of manipulation apparatus 3500 may include, for example, a pair of spaced-apart shoulder supports configured to engage the shoulders of the patient such that base supports 3510 are substantially fixed relative to the torso of the patient. Each base support 3510 is coupled to a linkage 3512. In aspects, each base support 3510 is pivotably coupled to the corresponding linkage 3512 via a pivot 3514, e.g., about an axis substantially parallel to the frontal plane of the patient and substantially perpendicular to the median plane of the patient.

Outer frame 3520 of manipulation apparatus 3500 includes a pair of substantially parallel, spaced-apart outer frame legs 3522 each defining first and second end portions 3523a, 3523b, respectively. First end portions 3523a of outer frame legs 3522 are connected to one another by a crossbar 3524 of outer frame 3520. Second end portions 3523b of outer frame legs 3522 are connected to respective base supports 3510 via linkages 3512. More specifically, second end portions 3523b of outer frame legs 3522 are coupled to linkages 3512, in aspects, via a slider mechanism 3526 enabling sliding of base supports 3510 towards and away from outer frame 3520. As an alternative to linkages 3512 coupling base supports 3510 and outer frame legs 3522 with one another, base supports 3510 and outer frame legs 3522 may be directly coupled to one another or coupled in any other suitable manner that enables pivoting and/or sliding of base supports 3510 relatives to outer frame legs 3522.

Continuing with reference to FIGS. 35-37B, inner frame 3530 of manipulation apparatus 3500 includes a pair of substantially parallel, spaced-apart inner frame legs 3532 each defining first and second end portions 3533a, 3533b, respectively. First end portions 3533a of inner frame legs 3532 pivotably support a head frame 3534 therebetween, e.g., about pivots 3535, thus enabling pivoting of head frame 3534 relative to inner frame legs 3532. Head frame 3534, also referred to as a halo, is configured to engage the head of the patient and extend circumferentially about the head of the patient. Second end portions 3533b of inner frame legs 3532 are coupled to corresponding outer frame legs 3522 via a pin and slot mechanism 3536 enabling pivoting and sliding of inner frame legs 3532 relative to outer frame 3510.

The above-detailed configuration of manipulation apparatus 3500 enables: pivoting of the entire head and neck of the patient anteriorly or posteriorly relative to the torso of the patient, e.g., via the pivotable connection of base supports 3510 with outer frame 3520; pivoting of a superior portion of the head and neck of the patient anteriorly or posteriorly relative to the torso of the patient, e.g., via the pivotable connection of inner frame 3530 with outer frame 3520; pivoting of the head of the patient anteriorly or posteriorly relative to the neck of the patient, e.g., via the pivotable connection of head frame 3534 with inner frame legs 3532; and extension or compression of the head and neck of the patient relative to the torso of the patient, e.g., via the slidable connection of linkages 3512 with outer frame 3520 and/or the slidable connection of inner frame 3530 with outer frame 3520. With respect to this extension, traction may be applied to head frame 3534, e.g., as indicated by force vector “V,” to provide extension. The traction can be maintained to thereby maintain the extension, or manipulation apparatus 3500 may be locked to maintain the extension.

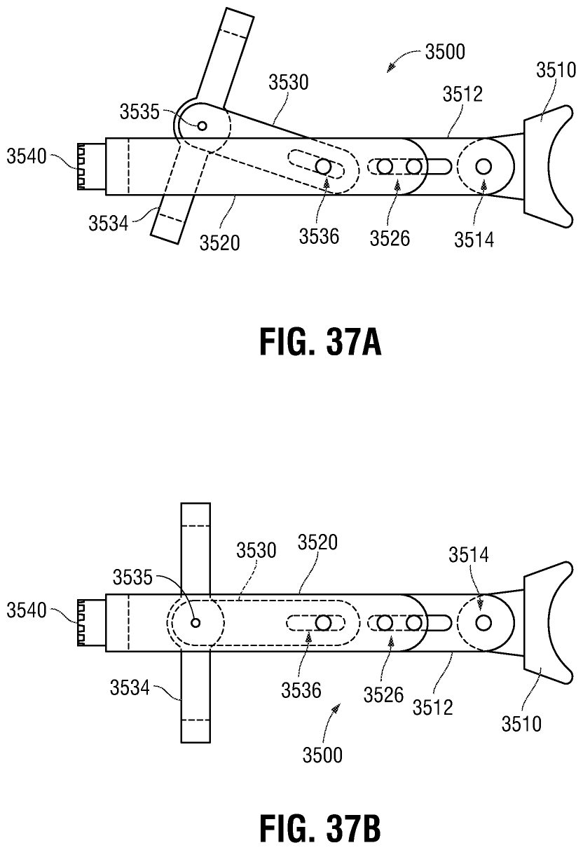

In aspects, the pivoting axis about which head frame 3534 pivots relative to inner frame legs 3532, the pivot axis about which inner frame legs 3532 pivot relative to outer frame 3510, and the pivot axis about which base supports 3510 pivot relative to outer frame 2510 are substantially parallel to one another (see FIGS. 37A and 37B). Further, in aspects, suitable locking mechanisms, e.g., ratchets, set screws, clamps, latches, splined connectors, and/or other suitable locking mechanisms, may be provided for selectively locking one or more of the above-detailed ranges of motion of manipulation apparatus 3500, thus enabling selective manipulation and subsequent fixed positioning of the patient.

Referring still to FIGS. 35-37B, the one or more mounting extensions 3540 enable mounting of manipulation apparatus 3500 to a surgical table, bed, or other support (including a Mayfield positioner) with outer frame 3520 fixed relative to the surgical support. More specifically, the one or more mounting extensions 3540 are engaged with crossbar 3524 of outer frame 3520 and extend therefrom to enable connection with a mount 3560 (see FIG. 35) of the support. In aspects, a plurality of mounting extensions 3540 are provided including a mounting extension 3540 aligned along a central longitudinal axis of manipulation apparatus 3500 and one or more mounting extensions 3540 offset on a first side of the central longitudinal axis of manipulation apparatus 3500 and/or offset on a second, opposite side of the central longitudinal axis of manipulation apparatus 3500. Thus, with respect to a mount 3560 (see FIG. 35) of a surgical support that is centered on the longitudinal axis of the support, manipulation apparatus 3500 may be utilized with the patient centered on the longitudinal axis of the support, offset to one side of the longitudinal axis of the support, or offset to the other side of the longitudinal axis of the support. As noted above, positioning the patient offset from the longitudinal axis of the support may be advantageous in robotic-assisted ACDF procedures and/or other procedures utilizing robotics such as, for example, to position the patient the closer to the surgical robot “R” to ensure reachability of the arm of the surgical robot “R” (see FIG. 1).

In aspects, manipulation apparatus 3500 (e.g., base supports 3510, outer frame 3520, and/or head frame 3534 thereof) may provide a mounting point for mounting one or more surgical components such as, for example: a navigation component, e.g., an emitter array of a navigation system; a surgical retractor assembly; a surgical robot, surgical robot or tool port; or surgical robot or tool holder; etc.

Manipulation apparatus 3500, in aspects, may mount to a surgical support and extend from an end or side of the support such that manipulation apparatus 3500 supports at least a portion of the patient without the support below the patient (or without the support directly otherwise directly supporting that portion of the patient). Further as noted above, manipulation apparatus 3500 may be detached from the support to enable the manipulation apparatus 3500 to be worn by the patient, e.g., to facilitate recovery from surgery and/or for other purposes.

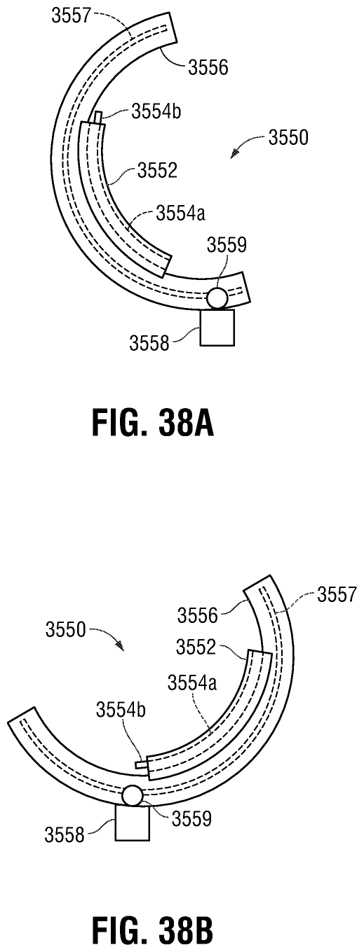

With reference to FIGS. 38A and 38B, in conjunction with FIG. 35, as noted above, manipulation apparatus 3500 includes an adjustable bolster assembly 3550. Adjustable bolster assembly 3550 is configured to rotate a bolster 3552 about the longitudinal axis of the patient (or an axis substantially parallel to the longitudinal axis of the patient) to enable positioning of the bolster 3552 to support the patient at different locations about the patient. In aspects, bolster 3552 is further configured to translate along the longitudinal axis of the patient (or an axis substantially parallel to the longitudinal axis of the patient) to support the patient at different locations along the patient. Bolster 3552, in aspects, may including an inflatable bladder 3554a connected to a port 3554b configured to connect to a fluid source to enable selective inflation (and deflation) of bolster 3552 to a desired configuration.

Bolster assembly 3550 further includes an arm 3556 defining a substantially C-shaped configuration and a base 3558. Arm 3556 supports bolster 3552 thereon and includes a track 3557. Base 3558 slidably supports arm 3556 and includes a guide 3559 received within track 3557 to confine slidable movement of arm 3556 relative to base 3558 along a determined path, e.g., an arc. Thus, arm 3556 may be moved relative to base 3558 (with guide 3559 moving along track 3557) to thereby reposition bolster 3552 relative to base 3558. In aspects, base 3558 is engaged with outer frame 3520 of manipulation apparatus 3500 such that repositioning bolster 3552 relative to base 3558 repositions bolster 3552 relative to outer frame 3520. In aspects, a locking mechanism is provided to selectively lock bolster 3552 in position relative to outer frame 3520.

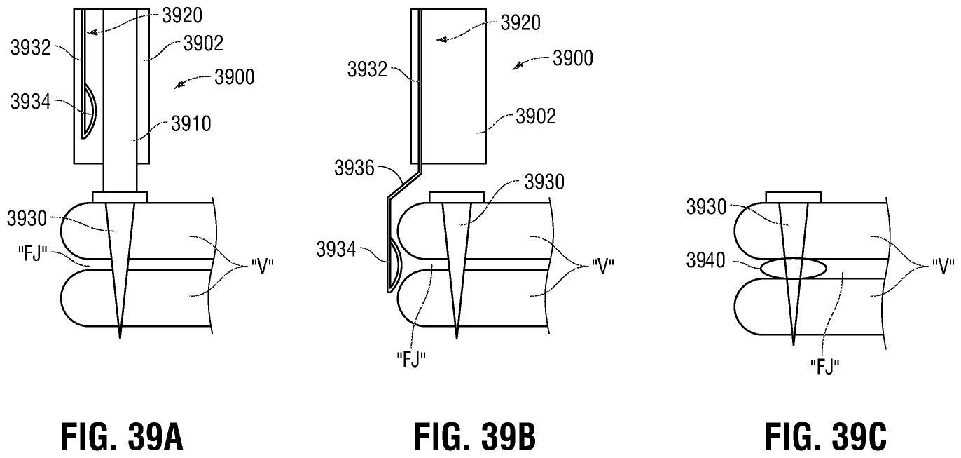

Referring to FIGS. 39A-39C, in aspects, a single access location may be utilized to provide access to guide surgical tools, devices, and/or materials to a surgical site, for example, posterior portions of a patient's spinal anatomy, e.g., a facet joint “FJ” and/or posterior bone surface. More specifically, a cannula 3900 may be configured to permit passage of a first surgical instrument 3910 through a longitudinal lumen 3902 thereof to, for example, introduce a transfacet screw 3930 through facet bones of adjacent vertebral bodies “V” to immobilize the facet joint “FJ.” Cannula 3900 may be oriented substantially transverse to the facet joint “FJ” for screw insertion, although other orientations are also contemplated.

Cannula 3900 also enables passage of a second surgical instrument 3920 configured to prepare, e.g., rough, bleed, decorticate, etc., the opposing bone surfaces of the facet joint “FJ” to facilitate insertion of an insert 3940 (FIG. 39C), e.g., graft material, into the facet joint “FJ.” Second surgical instrument 3920 may be inserted together with first surgical instrument 3910, prior to insertion of first surgical instrument 3910, or after removal of first surgical instrument 3910. Thus, the facet joint “FJ” may be prepared for the insert 3940 and/or the insert 3940 installed prior to or after screw fixation, e.g., using screw 3930 (FIGS. 39B and 39C). In other aspects, insert installation is performed as an alternative to screw fixation.

Second surgical instrument 3920 includes a shaft 3932 and an end effector 3934 disposed at a distal end of shaft 3932. End effector 3934 may be configured as a rasp, shaver, burr, and/or other suitable surgical instrument configured to prepare bone surfaces for insertion of graft material or other suitable insert. In aspects, shaft 3932 includes a movable portion 3936 disposed along a length thereof and/or connecting shaft 3932 with end effector 3934. Movable portion 3936 may include a flexible, shape memory material configured to permit insertion of shaft 3932 through cannula 3900 in a constrained configuration of shaft 3932 (see FIG. 39A) and, upon emergence of shaft 3932 from cannula 3990, to return to an unconstrained configuration (see FIG. 39B) wherein at least a portion of shaft 3932 extends beyond the outer peripheral volume defined by cannula 3900. In the unconstrained configuration (FIG. 39B), for example, movable portion 3936 of shaft 3932 may include one or more angles and/or curves such that end effector assembly 3934 is laterally displaced relative to cannula 3900, thus enabling access to the opposing bone surfaces of the facet joint “FJ.” As an alternative to shape memory material, movable portion 3936 may include one or more joints configured to, passively or actively (e.g., with steering), enable movement of movable portion 3936 from the constrained configuration to the unconstrained configuration. In aspects, shaft 3932 is substantially linear in the constrained configuration.

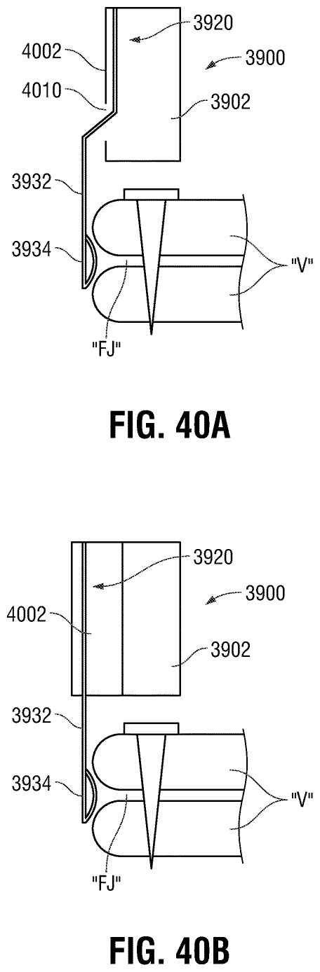

Turning to FIGS. 40A and 40B, in aspects, cannula 3900 may include features to facilitate positioning second surgical instrument 3920 to access the opposing bone surfaces of the facet joint “FJ.” More specifically, as shown in FIG. 40A, cannula 3900 may define a window 4010 through a side wall 4020 thereof and disposed in communication with longitudinal lumen 3902 to enable end effector 3934 and shaft 3932 of second surgical instrument 3920 to exit longitudinal lumen 3902 of cannula 3900 through window 4010, thus facilitating advancement of end effector 3934 to a location laterally spaced from cannula 3900, e.g., to access the opposing bone surfaces of the facet joint “FJ.”

As shown in FIG. 40B, cannula 3900 may additionally or alternatively include, in addition to longitudinal lumen 3902, e.g., formed by a first tube, a second longitudinal lumen 4002, e.g., formed by a second tube. Second longitudinal lumen 4002 may be fixed relative to longitudinal lumen 3902 in substantially parallel orientation relative thereto or at an angle relative thereto. Longitudinal lumen 3902 enables passage of first surgical instrument 3910 (FIG. 39A), as detailed above, while longitudinal lumen 4002 enables passage of second surgical instrument 3920. Second surgical instrument 3920, in aspects where a second lumen 4002 is provided, may be movable between the constrained and unconstrained configurations as detailed above or may be fixed, whereby the positioning of second lumen 4002 relative to first lumen 3902 enables access to the opposing bone surfaces of the facet joint “FJ.”

In aspects, a third surgical instrument similar to the second surgical instrument 3920 may be provided to install the insert, e.g., graft material, within the facet joint “FJ,” e.g., by insertion laterally between the opposing bone surfaces. That is, rather than having an end effector for preparing the bone surfaces as with second surgical instrument 3920, the third surgical instrument includes an end effector configured to install the insert, e.g., deploy the graft material.

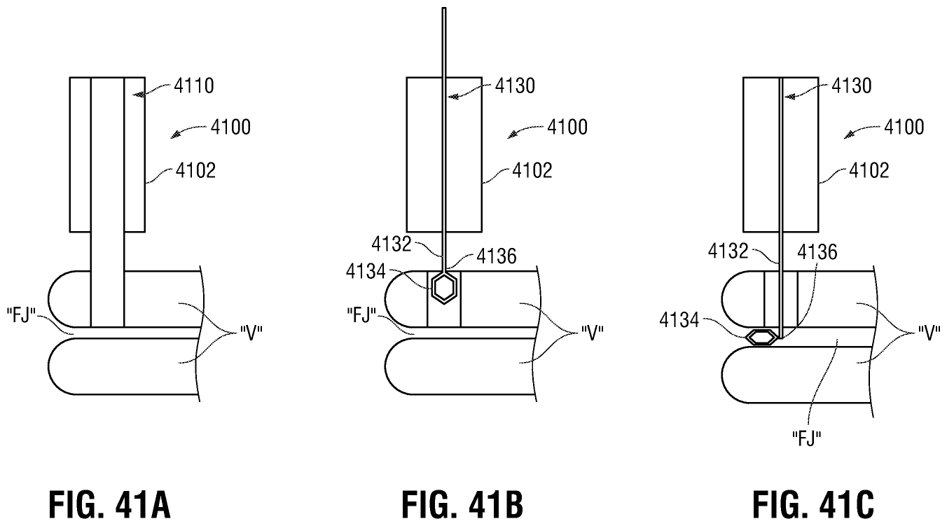

With reference to FIGS. 41A-41C, another configuration wherein a single access location is utilized to provide access to guide surgical tools, devices, and/or materials to a surgical site, for example, posterior portions of a patient's spinal anatomy, e.g., a facet joint “FJ” and/or posterior bone surface of adjacent vertebral bodies “V,” is detailed. More specifically, a cannula 4100 may be configured to permit passage of a first surgical instrument 4110 through a longitudinal lumen 4102 thereof to, for example, drill through the facet bone of one of the adjacent vertebral bodies “V” cooperating to define the facet joint “FJ,” thus providing access to the facet joint “FJ.” Thereafter, a second surgical instrument 4130 may be inserted through longitudinal lumen 4102 and the drilled hole in the vertebral body “V” to access the facet joint “FJ.”

Second surgical instrument 4130 includes a shaft 4132 and an end effector 4134, which may be configured as a rasp, shaver, burr, and/or other suitable surgical instrument configured to prepare bone surfaces for insertion of graft material or other suitable insert. In aspects, shaft 4132 includes a movable portion 4136 disposed along a length thereof and/or connecting shaft 4132 with end effector 4134. Movable portion 4136 may include, for example, one or more pivot joints configured to permit pivoting of end effector 4134 between an aligned configuration, wherein end effector 4134 is substantially aligned with shaft 4132 to facilitate insertion of second surgical instrument 4130 through cannula 4100, the drilled hole in the vertebral body “V,” and into the facet joint “FJ” to prepare the opposing bone surfaces. Thereafter, in aspects, a screw 3930 (FIGS. 39B and 39C) may be inserted through cannula 4100, the drilled hole in the vertebral body “V,” the facet joint “FJ,” and the opposing bone of the other vertebral body “V” to complete the fixation. In other aspects, the screw 3930 (FIGS. 39B and 39C) is omitted.

Referring generally to FIGS. 39A-41C, in aspects, the screws may be installed on either or both sides of the vertebral bodies and may be installed in spaced apart orientation relative to one another or in a crossing or intersecting orientation. Furter, in aspects, at least portions of the screws are cannulated to define a central passage and fenestrated openings to permit graft material to be inserted into the facet joint “FJ” through the screws. The screws may additionally or alternatively include washers, plates, and/or any other features for positioning at the heads of the screws to inhibit back out. For example, a plate on the head of the screw may be configured to contact the spinous process and/or an anti-rotation tab configured to engage bone may be provided to inhibit the screws from backing out.

Continuing with reference to FIGS. 39A-41C, any or all of the surgical instruments detailed may be handheld surgical instruments or may be robotic surgical instruments. Further, navigation may be utilized for handheld surgical instruments and/or robotic surgical instruments to facilitate screw fixation and/or implant installation.

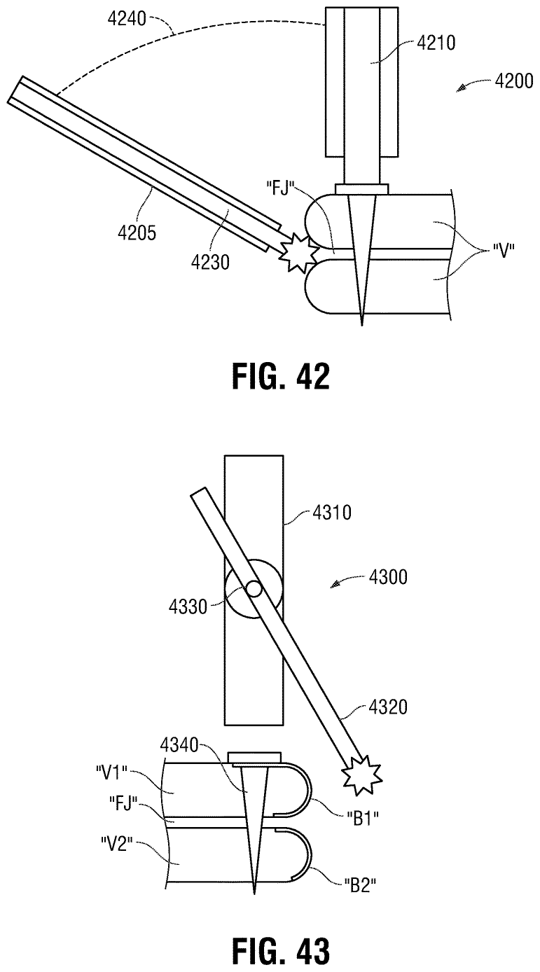

Turning to FIG. 42, in aspects, rather than a single access location, multiple access locations may be utilized to guide surgical tools, devices, and/or materials to posterior portions of a patient's spinal anatomy, e.g., a facet joint “FJ” and/or posterior bone surface. For example, a first surgical instrument 4210 may be inserted through a first cannula 4200 disposed at a first location and orientation to introduce a transfacet screw 4220 through facet bones of adjacent vertebral bodies “V” to immobilize the facet joint “FJ,” and a second surgical instrument 4230 may be inserted through a second cannula 4205 disposed at a second location and orientation to enable preparation, e.g., roughening, bleeding, decorticating, etc., of the opposing bone surfaces of the facet joint “FJ.” The second cannula 4205 may also be utilized for insertion of an insert, e.g., graft material, into the facet joint “FJ,” although the insert may alternatively be installed through the first cannula 4200.

In aspects, the first and second cannulae 4200, 4205 may be coupled via a coupling assembly 4240 configured to clock first and second cannulae 4200, 4205 at fixed orientations relative to one another, similarly as detailed above with respect to FIGS. 24A-25. Alternatively, first and second cannulae 4200, 4205 (and/or first and second surgical instruments 4210, 4230) may be engaged with robotic arms of a surgical robot to clock their orientations relative to one another. Further, the orientation of one of first or second cannula 4200, 4205 (and/or first or second surgical instrument 4210, 4230) may be utilized as a reference for navigation of the other.

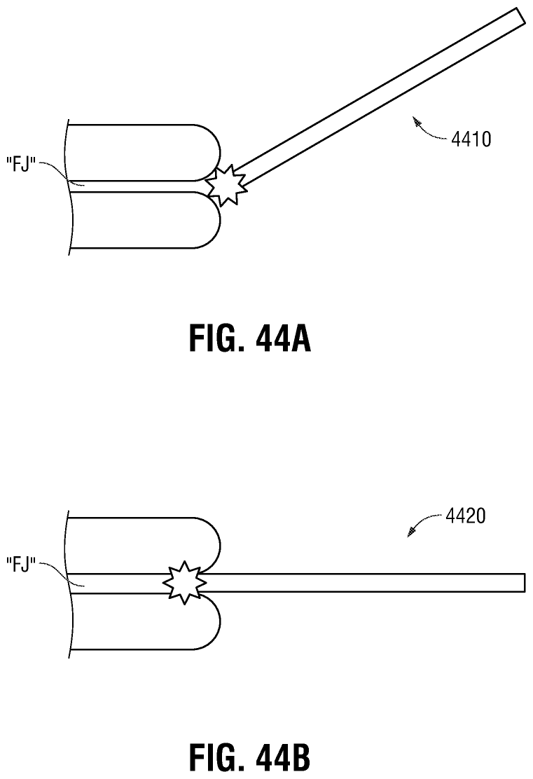

With reference to FIG. 43, a cannula assembly 4300 provided in accordance with the present disclosure includes a first cannula 4310 and a second cannula 4320 pivotably coupled to first cannula 4310 about a pivot 4330. More specifically, second cannula 4320 is pivotably relative to first cannula 4310 about pivot 4330 between an aligned orientation, wherein first and second cannulae 4310, 4320 are substantially parallel or coaxial with one another, and an angled orientation wherein second cannula 4320 is angled relative to first cannula 4310 and protrudes beyond the outer dimensions of first cannula 4310 at least at the distal end of first cannula 4310.

First cannula 4310 may be configured to permit passage of a first surgical instrument to introduce a transfacet screw 4340 through facet bones of adjacent vertebral bodies “V1,” “V2” to immobilize the facet joint “FJ.” First cannula 4310 may thus be oriented substantially transverse to the facet joint “FJ” for screw insertion, although other orientations are also contemplated.

Second cannula 4320, as noted above, is pivotable relative to first cannula 4310, thus providing an angled orientation for insertion of a second surgical instrument 4350 configured to prepare, e.g., rough, bleed, decorticate, etc., the opposing bone surfaces of the facet joint “FJ” to facilitate insertion of an insert, e.g., graft material, into the facet joint “FJ.” Second cannula 4320, more specifically, may be oriented to enable second surgical instrument 4350 to access to the facet joint “FJ.” The insert may be provided through second cannula 4320, first cannula 4310, or in another other suitable manner. Second surgical instrument 4350 may be configured as a rasp, shaver, burr, and/or other suitable surgical instrument configured to prepare bone surfaces for insertion of graft material or other suitable insert. In aspects, pivot 4330 is selectively lockable to fix the orientations of first and second cannulae 4310, 4320 relative to one another.

Continuing with reference to FIG. 43, and without limitation to use of cannula assembly 4300, in aspects, navigation may be utilized to plan the preparation of the opposing bone surfaces of the facet joint “FJ” to enable a surgical robot to operate second surgical instrument 4350 to perform the preparation. An installed screw, cannula 4310, cannula 4320, and/or other suitable reference may be utilized to facilitate navigation. More specifically, boundaries along the bone surfaces and/or depth boundaries may be defined and, thereafter, second surgical instrument 4350 robotically navigated and controlled to prepare the bone surfaces within the defines boundaries. For example, a first boundary “B1” may be defines over a first area of the first vertebral body “V1” while a second boundary “B2” is defined over a second area of the second vertebral body “V2.” The first and second boundaries “B1,” B2” may have different depths from one another and/or varied depths themselves. Thus, second surgical instrument 4350 robotically navigated and controlled to precisely prepare the bone surfaces according to the defined boundaries ‘B1,” B2.”

Referring to FIGS. 44A and 44B, depending upon whether only an insert, e.g., graft, is utilized for transfacet fixation, or whether both an insert and screw are utilized for transfacet fixation, the trajectory and surgical instrument utilized to prepare the surfaces surrounding the facet joint “FJ” may vary. For example, as shown in FIG. 44A, where only an insert is utilized, a relatively larger surgical instrument 4410, e.g., surgical burr of a first diameter, is used to burr a defect in bone at an angle (or steeper angle) relative to the facet joint “FJ.” On the other hand, as shown in FIG. 44B, where both an insert and screw are utilized, a relatively smaller surgical instrument 4420, e.g., surgical burr of a second, smaller diameter, is used to burr a defect in bone substantially parallel to (or at a shallow angle relative to) the facet joint “FJ.” In aspects, the relatively larger surgical instrument 4410 is a burr having a diameter of about 7.5 mm (or from about 6 mm to about 10 mm) and/or the relatively smaller surgical instrument 4420, is a burr having a diameter of about 2 mm (or from about 1.5 mm to about 3.0 mm).

Aspects of this disclosure may be further described by reference to the following numbered paragraphs:

1. A method of single position 360 degree cervical fusion, comprising: positioning a patient on a surgical table in a lateral position; orienting and retaining a spine of the patient in a desired sagittal alignment; performing anterior cervical disc fusion (ACDF); reorienting and retaining a head of the patient to facilitate posterior access; and performing posterior fixation.

2. The method according to paragraph 1, wherein the posterior fixation includes transfacet screw fixation.

3. The method according to paragraph 2, wherein the transfacet screw fixation is performed with fusion.

4. The method according to paragraph 3, wherein the fusion includes removing facet joint cartilage and decorticating a facet joint surface.

5. The method according to paragraph 4, wherein the fusion further includes delivering a flowable orthobiologic to a transfacet joint space.

6. The method according to paragraph 2, wherein the transfacet screw fixation is performed without fusion.

7. The method according to paragraph 1, wherein the posterior fixation includes at least one of lateral mass screw fixation or pedicle screw fixation.

8. The method according to any one of paragraphs 1-7, wherein at least a portion of the ACDF is performed by a surgeon with handheld tools, and wherein at least a portion of the posterior fixation is performed with at least one of: a surgical robot or at least one robotically guided tool.

9. The method according to paragraph 8, wherein the surgeon is positioned on an anterior side of the patient and the surgical robot is positioned on a posterior side of the patient.

10. The method according to any one of paragraphs 1-9, wherein the ACDF includes installing an anterior plate retained by a plurality of screws.

11. The method according to paragraph 10, further comprising attaching a navigation component to the anterior plate and utilizing the navigation component during the posterior fixation.

12. The method according to any one of paragraphs 1-11, further comprising airplaning the surgical table at least one of before performing the ACDF or before performing the posterior fixation.

13. The method according to any one of paragraphs 1-12, wherein a manipulation apparatus attached to the patient is utilized to orient the spine of the patient, retain the spine of the patient, reorient the head of the patient, and retain the head of the patient.

14. The method according to any one of paragraphs 1-13, wherein positioning the patient on the surgical table in the lateral position includes extending an upward arm of the patient along a body of the patient and extending a downward arm of the patient anteriorly from the body and bent at the elbow such that a lower portion of the downward arm of the patient extends cephalad.

15. The method according to any one of paragraphs 1-14, wherein performing the posterior fixation includes at least one of: planning a trajectory of at least one screw or installing at least one screw using navigation.

16. The method according to any one of paragraphs 1-15, wherein performing the ACDF includes retracting a wound with first and second retractor blades arranged in a vertical orientation.

17. The method according to any one of paragraphs 1-16, further comprising providing additional support to a posterior side of the patient during at least a portion of the ACDF.

18. The method according to paragraph 17, further comprising removing the additional support after the at least the portion of the ACDF.

19. The method according any one of paragraphs 1-18, wherein positioning the patient on the surgical table includes positioning the patient offset from a longitudinal axis of the surgical table.

20. The method according to any one of paragraphs 1-19, wherein positioning the patient on the surgical table includes securing the patient relative to the surgical table.

21. A manipulation apparatus configured to facilitate positioning and maintaining a spine of a patient, the manipulation apparatus comprising: a first frame assembly including a base frame configured to engage a torso of a patient to fix the base frame relative to the torso of the patient; and a second frame assembly coupled to the first frame assembly, the second frame assembly including a support frame and a head frame coupled to the support frame and configured to engage a head of the patient to fix the head frame relative to the head of the patient, wherein the support frame is movable relative to the base frame in at least two degrees of freedom, and wherein the head frame is movable relative to the support frame in at least one degree of freedom.

22. The manipulation apparatus according to paragraph 21, wherein the support frame is configured to pivot relative to the base frame to enable pivoting of the head and neck of the patient relative to the torso of the patient and to translate relative to the base frame to enable extension or compression of the head and neck of the patient relative to the torso of the patient.

23. The manipulation apparatus according to paragraph 22, wherein the support frame is configured to lock relative to the base frame to at least one of retain a position of the head and neck of the patient relative to the torso of the patient or retain an extension or compression of the head and neck of the patient relative to the torso of the patient.

24. The manipulation apparatus according to any one of paragraphs 21-23, wherein the head frame is configured to pivot relative to the support frame to enable pivoting of the head of the patient anteriorly or posteriorly relative to the neck of the patient.

25. The manipulation apparatus according to paragraph 24, wherein the head frame is configured to lock relative to the support frame to retain the head of the patient relative to the neck of the patient.

26. The manipulation apparatus according to any one of paragraphs 21-25, wherein the second frame assembly further includes an outer frame coupling the support frame with the first frame assembly.

27. The manipulation apparatus according to paragraph 26, wherein the first frame assembly further includes a pair of linkages coupling the outer frame with the base frame.

28. The manipulation apparatus according to paragraph 26 or 27, wherein the outer frame includes at least one mounting extension configured to enable mounting of the outer frame to a patient support.

29. The manipulation apparatus according to paragraph 26, wherein the outer frame includes a plurality of mounting extensions, at least one mounting extension of the plurality of mounting extensions configured to enable mounting of the outer frame to a patient support in an offset position.

30. The manipulation apparatus according to any one of paragraphs 21-29, further comprising an adjustable bolster assembly coupled to the second frame assembly, wherein the adjustable bolster assembly includes a bolster that is movable relative to the second frame assembly.

31. A surgical periscope, comprising: a body defining a first end portion and a second end portion, the first end portion of the body configured to operably couple to an objective end of a surgical microscope having a first viewing direction, the second end portion of the body disposed at an angle relative to the first end portion of the body and having a second viewing direction disposed at an angle relative to the first viewing direction; and at least one optical element disposed within the body and configured to relay a field of view in the second viewing direction to the objective end of the surgical microscope to enable visualization of the field of view through the surgical microscope.

32. The surgical periscope according to paragraph 31, wherein the first end portion of the body is configured to releasably engage the surgical microscope at the objective end of the surgical microscope.

33. The surgical periscope according to paragraph 31 or 22, wherein the first end portion of the body is one of: physically connected to the second end portion of the body or wirelessly connected to the second end portion of the body.

34. The surgical periscope according to any one of paragraphs 31-33, wherein the second viewing direction is disposed at an angle of about 90 degrees relative to the first viewing direction.

35. A support assembly for supporting a patient in a lateral position on a surgical table, the support assembly comprising: a mount configured for positioning on a surgical table; and a first support rotatably coupled to the mount, the first support including a body having a plurality of independently adjustable sections to enable independent adjustment of a height of each of the plurality of independently adjustable sections.

36. The support assembly according to paragraph 35, further comprising a strap attached to the first support and configured to strap a head of a patient to the first support.

37. The support assembly according to paragraph 35 or 36, further comprising a second support pivotably coupled to the first support, the second support defining an elongate configuration.

38. A method of single position 360 degree cervical fusion, comprising: positioning a patient on a surgical table in a lateral position; performing anterior cervical disc fusion (ACDF); and performing posterior fixation, wherein the posterior fixation includes: from a first approach to a facet joint, at least one of: preparing for insertion of an insert into the facet joint or inserting the insert into the facet joint; and from a second, different approach to the facet joint, performing transfacet screw fixation.

39. The method according to paragraph 38, wherein at least one first instrument is used for the first approach and at least one second instrument is used for the second approach, the first and second instruments coupled to one another in at least one degree of freedom.

40. The method according to paragraph 38 or 39, wherein the first approach is adjacent to opposing bone surfaces of the facet joint and wherein the second approach is transverse to the facet joint.

While several aspects of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

1-15. (canceled)

16. A method of single position 360 degree cervical fusion, comprising:
positioning a patient on a surgical table in a lateral position;
orienting and retaining a spine of the patient in a desired sagittal alignment;
performing anterior cervical disc fusion (ACDF);
reorienting and retaining a head of the patient to facilitate posterior access; and
performing posterior fixation.

17. The method according to claim 16, wherein the posterior fixation includes transfacet screw fixation.

18. The method according to claim 17, wherein the transfacet screw fixation is performed with fusion.

19. The method according to claim 18, wherein the fusion includes removing facet joint cartilage and decorticating a facet joint surface.

20. The method according to claim 19, wherein the fusion further includes delivering a flowable orthobiologic to a transfacet joint space.

21. The method according to claim 17, wherein the transfacet screw fixation is performed without fusion.

22. The method according to claim 16, wherein the posterior fixation includes at least one of lateral mass screw fixation or pedicle screw fixation.

23. The method according to claim 16, wherein at least a portion of the ACDF is performed by a surgeon with handheld tools, and wherein at least a portion of the posterior fixation is performed with at least one of: a surgical robot or at least one robotically guided tool.

24. The method according to claim 23, wherein the surgeon is positioned on an anterior side of the patient and the surgical robot is positioned on a posterior side of the patient.

25. The method according to claim 16, wherein the ACDF includes installing an anterior plate retained by a plurality of screws.

26. The method according to claim 25, further comprising attaching a navigation component to the anterior plate and utilizing the navigation component during the posterior fixation.

27. The method according to claim 16, further comprising airplaning the surgical table at least one of before performing the ACDF or before performing the posterior fixation.

28. The method according to claim 16, wherein a manipulation apparatus attached to the patient is utilized to orient the spine of the patient, retain the spine of the patient, reorient the head of the patient, and retain the head of the patient.

29. The method according to claim 16, wherein positioning the patient on the surgical table in the lateral position includes extending an upward arm of the patient along a body of the patient and extending a downward arm of the patient anteriorly from the body and bent at the elbow such that a lower portion of the downward arm of the patient extends cephalad.

30. The method according to claim 16, wherein performing the posterior fixation includes at least one of: planning a trajectory of at least one screw or installing at least one screw using navigation.

31. The method according to claim 16, wherein performing the ACDF includes retracting a wound with first and second retractor blades arranged in a vertical orientation.

32. The method according to claim 16, further comprising providing additional support to a posterior side of the patient during at least a portion of the ACDF.

33. The method according to claim 32, further comprising removing the additional support after the at least the portion of the ACDF.

34. The method according to claim 16, wherein positioning the patient on the surgical table includes positioning the patient offset from a longitudinal axis of the surgical table.

35. The method according to claim 16, wherein positioning the patient on the surgical table includes securing the patient relative to the surgical table.
